(19) United States
(12) Patent Application Publication
(10) Pub. No.: US 2022/0296647 A1
(43) Pub. Date: Sep. 22, 2022

(54) DIFFERENTIATION INDUCER CONTAINING NUCLEUS PULPOSUS PROGENITOR CELL MASTER REGULATOR TRANSCRIPTION FACTORS, METHOD FOR PRODUCING INDUCED NUCLEUS PULPOSUS PROGENITOR CELLS, AND USE OF INDUCED NUCLEUS PULPOSUS PROGENITOR CELLS

(71) Applicant: Tokai University Educational System, Tokyo (JP)

(72) Inventors: Daisuke SAKAI, Kanagawa (JP); Jordy SCHOL, Kanagawa (JP); Yoshihiko NAKAMURA, Kanagawa (JP)

(73) Assignee: Tokai University Educational System, Tokyo (JP)

(21) Appl. No.: 17/599,396

(22) PCT Filed: Jul. 29, 2021
(86) PCT No.: PCT/JP2021/028084

§ 371 Date: Sep. 28, 2021

(30) Foreign Application Priority Data
Sep. 29, 2020 (JP) 2020-163416

(51) Int. Cl. A61K 35/32; C12N 5/077

(52) U.S. Cl. CPC A61K 35/32; C12N 5/0655; C12N 2501/60; C12N 2506/1307; C12N 2506/1353; C12N 2501/115

(57) ABSTRACT

Provided is reproducible means that enables production of nucleus pulposus progenitor cells (preferably, an active nucleus pulposus progenitor cell phenotype) from desired cells such as terminally differentiated cells and stem cells having pluripotency or multipotency. A nucleus pulposus progenitor cell inducer according to the present invention comprising an effective amount of a gene of Brachyury (T) or a homolog thereof, at least one selected from the group consisting of SRY-box6 (SOX6) or a homolog thereof and Forkhead Box Q1 (FOXQ1) or a homolog thereof, and MYC Proto-Oncogene, BHLH Transcription Factor (cMyc) or a homolog thereof (nucleus pulposus progenitor cell master regulator transcription factor), or a product thereof.

### TECHNICAL FIELD

The present invention relates to transcription factors (nucleus pulposus cell master regulator transcription factors) that enable cell reconstruction (direct reprogramming) from terminally differentiated cells or cells with differentiation capacity to an active nucleus pulposus cell phenotype, that is, transdifferentiation or differentiation induction from undifferentiated cells to nucleus pulposus cells. The present invention particularly relates to nucleus pulposus progenitor cell master regulator transcription factors with high cell proliferative capacity that can produce many nucleus pulposus cells (active nucleus pulposus cell phenotype) and enable direct reprogramming to nucleus pulposus progenitor cells. The present invention further relates to use of the nucleus pulposus progenitor cell master regulator transcription factors.

### BACKGROUND ART

Low back pain and neck pain are common health problems that affect an approximate 632 million people worldwide and are major causes of disability. The two disorders place significant social and economic burdens due to work disability and medical costs. Intervertebral disc degeneration, which is estimated to develop 20% of all low back pain cases, impairs the biomechanics along the spinal column and may lead to disc herniation, spinal canal stenosis, spondylolisthesis, and other spinal disorders. Intervertebral disc degeneration is an intervertebral disc disorder that is particularly characterized by irreversible degeneration of the extracellular matrix composition in the core of the intervertebral disc.

At present, there is no clinically effective treatment that enables recovery from such a degenerative state or that can stop the underlying pathogenesis. Therefore, there is a strong demand for the development of new treatments, Nevertheless, general understanding of intervertebral disc homeostasis, cell phenotype, and development and progression of the pathogenesis is poor, particularly as compared with the knowledge insight in the field of bone and articular cartilage.

An intervertebral disc is the fibrocartilage structure between each two vertebrae along the spine. The intervertebral disc is involved in distributing mechanical forces along the spinal column while imparting flexibility. This feature arises from the hydrostatic pressure established within an enclosed nucleus pulposus. The nucleus pulposus (NP) is composed mainly of proteoglycans and type II collagen fibers and absorbs a relatively large amount of water. The nucleus pulposus is laterally surrounded by a fibrocartilage layer called annulus fibrosus (AF). Finally, the intervertebral disc is covered by a thin layer of hyaline cartilage on the boundary with each vertebra, that is, an endplate.

It is suggested that native nucleus pulposus cells change their phenotype from vacuolated notochord cells to active nucleus pulposus cells, senescent cells, and fibrous nucleus pulposus cells, to form many heterogeneous cell populations in the nucleus pulposus. Such changes to different phenotypes seem to play a decisive role in the progression of intervertebral disc degeneration. “Active nucleus pulposus cell phenotype” may be defined as “a nucleus pulposus cell (phenotype), that either actively and anabolically contributes to the production of or remodeling of their extracellular matrix or otherwise is actively supporting other cells to do so. Due to aging and biological and mechanical stress (wear), the nucleus pulposus shows a gradual decrease in Tie2/GD2-positive progenitor cells (Non Patent Literature 1: Sakai et al., 2012), and the aging cells progressively switch to fibrous cells. These changes result in a shift from a proteoglycan-rich nucleus pulposus extracellular matrix into a fibrous structure, thereby deteriorating the hydrostatic pressure and other biomechanical characteristics of the intervertebral disc. A cascade of these events potentially causes low back pain and other spinal disorders. Conversely, intervertebral disc degeneration and related disorders are rarely seen in other animal models (such as pigs, mice, and rats). This is likely due to the facts that these animals maintain the original notochordal cell phenotype, and as such are able to maintain healthy intervertebral discs with an active phenotype of nucleus pulposus cells, which play a decisive role in preventing the intervertebral disc disorders. Accordingly, for treating human intervertebral disc degeneration or the like or alleviating pains and disorders, a strong research emphasis is placed on establishing strategies to repopulate or induce proliferation of endemic cell populations in the intervertebral disc tissue to actively produce and regenerative intervertebral disc matrix.

As part of such strategies, various studies show the advantage of transplanting active nucleus pulposus cells or chondrocytes into affected areas by intervertebral disc degeneration or the like (as reviewed in Non Patent Literature 2: Schol and Sakai, 2018), and it is regarded as a promising option for treatment of intervertebral disc degeneration or the like. Nevertheless, supply of active nucleus pulposus cells or the like for cell transplantation is insufficient both clinically and scientifically. Conventionally, the possibility of regenerating intervertebral discs by transplanting autologous or allogeneic donor nucleus pulposus cells that are isolated from an intervertebral disc surgically removed during surgery or chondrocytes that essentially have the ability to produce an extracellular matrix like the nucleus pulposus cells in the intervertebral disc environment has been explored. However, since the intervertebral discs or cartilage collected from donors by surgery or the like are often damaged due to diseases, trauma, aging, or the like, there is a possibility that nucleus pulposus cells or chondrocytes contained therein do not have sufficient effectiveness as transplantation regeneration materials (Non Patent Literature 1). Further, in the treatment method of transplanting nucleus pulposus cells, donor cells are reactivated and generated by in-vitro culture to prepare the final purified product of active nucleus pulposus cell populations, but the traits of nucleus pulposus cells are known to be lost (dedifferentiated) by culture, and it is a technical problem to amplify nucleus pulposus cells without dedifferentiation (Non Patent Literature 1).

As cells to be transplanted into the intervertebral disc, it is also conceivable to use stern cells capable of differentiating into nucleus pulposus cells, such as mesenchymal stromal cells or mesenchymal stem cells (MSCs) that are derived from bone marrow, placenta, fat, or the like, comparatively easily accessible, and available in a large amount. However, the hypoglycemia, hyperosmolarity, and hypoxic environment caused by avascular and progressive degeneration of an intervertebral disc is a significant obstacle to the survival and thriving of MSCs. Therefore, it is unknown to what extent the transplanted MSCs can actively produce and secrete matrix proteins or cytokines for reactivating surrounding cells to regenerate the intervertebral disc tissue for long-term effects, and thus clinical application of MSCs to intervertebral disc disorders is limited (Non Patent Literature 3: Fang, Z., et al., 2013). There are similar problems in multipotent stem cells other than mesenchymal stromal cells or mesenchymal stem cells, such as hematopoietic stem cells.

Therefore, a technique for artificially maintaining or inducing the traits (phenotype) of active nucleus pulposus cells is required, but information on transcription factors and their control, which is the key of the technique, is limited, and induction of the active nucleus pulposus cell phenotype itself in vitro has not been realized so far. Conventionally, the possibility of obtaining a nucleus pulposus cell-like phenotype as an alternative to actual nucleus pulposus cells by applying various stimuli to cells that seem promising among cells other than nucleus pulposus cells has been studied. As such candidate cells, induced pluripotent stem cells (iPS cells) are used other than MSCs, although less frequently. It is known that various cells can be obtained from such pluripotent or multipotent stem cells by differentiation induction under appropriate conditions. In order to obtain a nucleus pulposus cell-like phenotype from pluripotent or multipotent stein cells, stimulation by extracellular matrix compositions, oxygen partial pressure, mechanical or osmotic pressure, application of scaffold such as hydrogel, stimulation by growth factors or cytokines, stimulation by co-culture with other cells or culture supernatant, or combinations thereof are attempted. These studies have generally reported that the expression level of nucleus pulposus cell-related genes such as type II collagen (COL2), aggrecan (ACAN), and differentiation cluster 24 (CD24) can be enhanced, or the production of those proteins can be improved. Further, it has been also reported that cell encapsulation, aggregation, or direct transplantation in vivo causes deposition of an extracellular matrix similar to an NP-cell like extracellular matrix rich in proteoglycans and COL2, which is generally reported as regeneration of an intervertebral disc.

In general, there is a concern about the reproducibility in the method using growth factors. In order to elicit a desired response by applying growth factors, cells need to present the correct cell membrane-bound receptors that correspond to the growth factors. The presentation of appropriate receptors is highly specific to the cell type and donor, thus potentially causing problems in reproducibility of the induction procedure. Further, it is still unknown whether there is a possibility that cell transplantation in an environment with no or different growth factors (that is, in an environment in the original nucleus pulposus tissue or the like) can negatively alter the transplanted cell phenotype. Finally, continuous supplementation of growth factors accounts for a relatively expensive part of a culture process for producing or using induced nucleus pulposus cells, thereby further limiting the clinical applicability.

Only a few studies have explored the possibility of fabricating an active nucleus pulposus cell phenotype from pluripotent or multipotent cells, such as MSCs and iPSCs, or terminally differentiated cells other than nucleus pulposus cells by more direct manipulation to the gene expression profile, instead of adjusting the culture conditions as above, and information on transcription factors and their control, which is the key of such gene operation, has been exceptionally limited.

For example, Non Patent Literature 4 (Xu et al., 2016) discloses that bone morphogenetic protein 7 (BMP7) was overexpressed in rabbit bone marrow-derived MSCs, in order to stimulate differentiation into NP cells by enhancing secretion of BMP7, as a result of which, the expressions of COL2, ACAN, SOX9, keratin (KRT)-8, and KRT19 were enhanced at the mRNA level by monolayer culture two to three weeks after transfection. However, when a cell product obtained in this method was transplanted into a rabbit model with intervertebral disc degeneration, a beneficial effect was observed in both the sham control and the BMP7 transfection group 6 weeks and 12 weeks after transplantation, but the glycosaminoglycan/DNA ratio in the BMP7 overexpression group at the 12th week were merely slightly increased as compared with the sham control.

Non Patent Literature 5 (Chen et al., 2017) discloses that, by the same approach, an established factor WNT11 was overexpressed by lentivirus transduction in rat fat-derived MSCs, and an in-vitro evaluation thereof showed a slight but significant increase in COL2, ACAN, and SOX9 at, both the mRNA and protein levels, as compared with the sham control transfected with green fluorescent protein (GFP) for overexpression.

As another approach, Non Patent Literature 6 (Outani et al., 2013) discloses that introduction of SOX9, known as a master regulator transcription factor for forming cartilage cells (Non Patent Literature 7: Wright et al., 1995, and Non Patent Literature 8: Bi et al., 1999), enabled direct reprogramming of their cells into a chondrocyte phenotype.

Non Patent Literature 9 (Yang et al, 2011) discloses that a cell line overexpressing SOX9 was established in rat adipose tissue-derived MSCs by a leukemia virus-derived viral vector pseudotyped with vesicular stomatitis virus G-envelope glycoprotein. Further, it also discloses that, as a result of culturing the established cell line with or without addition of transforming growth factor β(TGFβ)-3, the ratios GAG/DNA and COL2/DNA changed, and such a result showed that the overexpression of SOX9 could induce a relative increase in the ratios of GAG and COL2 with supplementation of TGFβ3, conversely, manipulation by only any one of supplementation of TGFβ3 to the culture medium or transduction with SOX9 would not result in a significant improvement. However, the literature does not present any further evaluation regarding a nucleus pulposus cell phenotype.

Likewise, Non Patent Literature 10 (Sun et al. 2014) also discloses that rabbit bone marrow-derived MSCs were transduced with SOX9 through an adenoviral vector. In an in-vitro monolayer evaluation, the transduced cells clearly showed an obviously strong increase in expressions of ACAN, COL2, and SOX9 at the mRNA level but showed only a slight decrease in type I collagen (COL1), as compared with the GFP-control group. The same results were obtained from transduced cells cultured using chitosan-glycerophosphate gel. Finally, when transduced cells were transplanted into a rabbit model with induced intervertebral disc degeneration, both a GFP-expressing MSC-administered group and a SOX9-overexpressing MSC-administered group showed improved T2 intensity, which showed the advantage of MSC transplantation again, but the SOX9-overexpressing MSC-administered group showed a small but significant improvement as compared with the GFP-expressing cells. Likewise, the histological results also revealed a benefit by MSC transplantation with a slight increase due to the SOX9 overexpression, as shown by safranin-O staining (representing the content of proteoglycan), and COL2 targeted staining, in contrast to the GFP expression.

Non Patent Literature 11 (Hiramatsu et al., 2011) discloses that mouse adult dermal fibroblasts were differentiated using combinations of reprogramming factor c-MYC with transcription factors KLF4 and SOX9, and as a result of overexpression of their genes, expressions of ACAN and COL2 specific to chondrocytes could be stimulated, despite the mature differentiated state of starting cell populations.

Non Patent Literature 12 (Outani et al, 2013, by the same group as Non Patent Literature 11) also discloses evidence of successful reprogramming from fibroblasts to a chondrocyte phenotype by overexpression of KLF4, c-MYC and SOX9. This literature discloses that expressions of ACAN and COL2 were enhanced by application to dermal fibroblasts derived from humans. Further, it is also revealed that transplantation of the reprogrammed fibroblasts enhanced deposition of a chondrogenic matrix in a mouse model with any one of subcutaneous and chondrogenic defect indications.

The aforementioned conventional art literatures generally indicate that expression of transcription factor SOX9 can stimulate the chondrogenic phenotype. Accordingly, overexpression of SOX9 highly possibly results in a more common chondrocyte phenotype rather than a specific nucleus pulposus cell phenotype. The conventional art literatures did not examine markers or morphological characteristics specifically associated with nucleus pulposus cells, as a comparison with other cells exhibiting cartilage traits.

In addition, as markers associated with the traits of nucleus pulposus cells, hypoxia-inducible factor 1 sub-unit α(HIF1α) (Non Patent Literature 13: Risbud et al., 2006), Brachyury (T) (Non Patent Literature 14: Sheyn et al., 2017), Paired Box 1 (PAX1) (Non Patent Literature 15: Risbud et al., 2015), and the like have been reported, but their gene expression profiles have not been sufficiently evaluated so far.

Non Patent Literature 16 (Sheyn D. et al., 2019) discloses a method for introducing iPS cells into a nucleus pulposus cell-like phenotype. However, this literature discloses that transcription factor manipulation, particularly, T overexpression is insufficient to induce and maintain a nucleus pulposus cell-like phenotype. Non Patent Literature 17 (Colombier P, et al., 2020) discloses a stepwise differentiation protocol, including overexpression of NOTO, T, and FOXA2, for producing nucleus pulposus cells from an iPS cell population. However, this literature discloses that T overexpression is insufficient to induce a nucleus pulposus cell-like phenotype. Non Patent Literature 18 (Zhang Y., et al., 2020) discloses a protocol for enabling differentiation from human iPS cells into nucleus pulposus cell-like cells (including nucleus pulposus progenitor cell-like cells) via stepwise differentiation including addition of components to a medium and detection of NOTO.

Meanwhile, Non Patent Literature 1 and Patent Literature 1 (WO 2011/122601) disclose that, of the cells contained in the intervertebral disc tissue (nucleus pulposus), cells positive for Tie2 and/or GD2 as a cell surface marker are cells to be called the stem cells or progenitor cells of nucleus pulposus cells (nucleus stern/progenitor cells), particularly, cells positive for both of Tie2 and GD2, that form spherical colonies, and finally are capable of differentiating into mature nucleus pulposus cells through a series of differentiation cascades (additionally capable of differentiating into adipocytes, osteocytes, chondrocytes, and nerve cells), and an extracellular matrix such as type II collagen can be produced in a tissue by transplantation of nucleus stem/progenitor cells into the intervertebral disc (nucleus pulposus), so that it may be possible to maintain or reconstruct the intervertebral disc tissue and prevent or treat degenerative disc diseases.

### CITATION LIST

### Patent Literature

[Patent Literature 1] WO 2011/122601

### Non Patent Literature

[Non Patent Literature 1] Sakai, D. et al. Exhaustion of nucleus pulposus progenitor cells with ageing and degeneration of the intervertebral disc. Nature communications 3, 1264, doi: 10.1038/ncomms 2226 (2012).

[Non Patent Literature 2] Schol Sakai D. Cell therapy for intervertebral disc herniation and degenerative disc disease: clinical trials. International orthopaedics, (2018) doi: haps://doi.org/10.1007/s00264-018-4223-1.

[Non Patent Literature 3] Fang, Z., et al., Differentiation of GFP-Bcl-2-engineered mesenchymal stem cells towards a nucleus pulposus-like phenotype under hypoxia in vitro. Biochem Biophys Res Commun, 2013. 432 (3): p. 444-50.

[Non Patent Literature 4] Xu, J., et al., BMP7 enhances the effect of BMSCs on extracellular matrix remodeling in a rabbit model of intervertebral disc degeneration. FEBS J, 2016. 283 (9): p. 1689-700.

[Non Patent Literature 5] Chen, H. T., et al., Wnt11 overexpression promote adipose-derived stem cells differentiating to the nucleus pulposus-like phenotype. Eur Rev Med Pharmacol Sci, 2017, 21 (7): p. 146:2-1470.

[Non Patent Literature 6] Outani, H. et al. Direct induction of chondrogenic cells from human dermal fibroblasts cultured by defined factors (PloS one, 2013).

[Non Patent Literature 7] Wright, E., et al., The Sry-related gene Sox9 is expressed during chondrogenesis in mouse embryos. Nat Genet, 1995. 9 (1): p, 15-20.

[Non Patent Literature 8] Bi, W., et al., Sox9 is required for cartilage formation. Nat Genet, 1999. 22 (1): p. 85-9.

[Non Patent Literature 9] Yang, Z., et al., Sox-9 facilitates differentiation of adipose tissue-derived stem cells into a chondrocyte-like phenotype in vitro. J Orthop Res, 2011. 29 (8): p. 1291-7.

[Non Patent Literature 10] Sun, W., et al., Sox9 gene transfer enhanced regenerative effect of bone marrow mesenchymal stem cells on the degenerated intervertebral disc in a rabbit model. PLoS One, 2014. 9 (4): p. e93570.

[Non Patent Literature 11] Hiramatsu, K., et al., Generation of hyaline cartilaginous tissue from mouse adult dermal fibroblast culture by defined factors. J Clin Invest, 2011. 121 (2): p, 640-57.

[Non Patent Literature 12] Outani, H., et al., Direct induction of chondrogenic cells from human dermal fibroblast culture by defined factors. PLoS One, 2013. 8 (10): p. e 77365.

[Non Patent Literature 13] Risbud, M. V., et al., Nucleus pulposus cells express HIF-1 alpha under normoxic culture conditions: a metabolic adaptation to the intervertebral disc microenvironment. JC ell Biochem, 2006. 98 (1): p. 152-9.

[Non Patent Literature 14] Sheyn et al (2017) Human iPS-derived notochordal cells survive and retain their phenotype in degenerated porcine IVD-ORS 2017 Annual Meeting Paper No.0051.

[Non Patent Literature 15] Risbud, M. V., et al., Defining the phenotype of young healthy nucleus pulposus cells: recommendations of the Spine Research Interest Group at the 2014 annual ORS meeting. J Orthop Res, 2015. 33 (3): p. 283-93.

[Non Patent Literature 16] Sheyn D, Ben-David S, Tawackoli W, et al. Human iPSCs can be differentiated into notochordal cells that reduce intervertebral disc degeneration in a porcine model. Theranostics. 2019; 9 (25): 7506-7524.

[Non Patent Literature 17] Colombier P, Halgand B, Chedeville C, et al. NOTO Transcription Factor Directs Human Induced Pluripotent Stem Cell-Derived Mesendoderm Progenitors to a Notochordal Fate. Cells. 2020; 9 (2).

[Non Patent Literature 18] Zhang Y, Zhang Z, Chen P, et al. Directed Differentiation of Notochord-like and Nucleus Pulposus-like Cells Using Human Pluripotent Stem Cells. Cell Rep. 2020; 30 (8): 2791-2806 e2795.

### SUMMARY OF INVENTION

### Technical Problem

As described above, there are various problems in transplanting intervertebral disc (nucleus pulposus) cells or chondrocytes collected from donors and also in transplanting stein cells capable of differentiating into nucleus pulposus cells such as mesenchymal stromal cells (mesenchymal stem cells) and hematopoietic stem cells, in treating intervertebral disc degeneration or the like. Further, there are also problems of reproducibility and cost in the method of differentiation induction of stem cells such as MSCs into an active nucleus pulposus cell phenotype using specific growth factors, and there is no evidence for achieving the safety or therapeutic effects that counteract such problems.

Therefore, it seems ideal to produce an active nucleus pulposus cell phenotype or nucleus pulposus progenitor cells that can induce differentiation into an active nucleus pulposus cell, by allowing specific transcription factors to act on stern cells or the like (that is, by changing the transcription factor profiles) and to use the active nucleus pulposus cell phenotype or nucleus pulposus progenitor cells thus obtained for transplantation. However, “nucleus pulposus cell master regulator transcription factors” or “nucleus pulposus progenitor cell master regulator transcription factors” that enable such production of cells, keep exerting sufficient functions as an active nucleus pulposus cell phenotype or nucleus pulposus progenitor cells also after transplantation of the active nucleus pulposus cell phenotype or nucleus pulposus progenitor cells obtained (such as an active nucleus pulposus cell phenotype producing a sufficient amount of extracellular matrix and nucleus pulposus progenitor cells producing a large number of active nucleus pulposus cell phenotypes), and thus can be clinically effective have not been established so far. Further, potent transcription factors (master regulator transcription factors) that enable an active nucleus pulposus cell phenotype or nucleus pulposus progenitor cells to be obtained by transdifferentiation from terminally differentiated cells other than nucleus pulposus cells, not from stem cells such as MSCs, have still not been found. SOX9, which has been established as a master regulator transcription factor for cartilage formation, has not been demonstrated to have sufficient usefulness as a master regulator transcription factor for an active nucleus pulposus cell phenotype or a nucleus pulposus progenitor cell.

It is an object of the present invention to provide reproducible means that enables the production of an active nucleus pulposus cell phenotype or nucleus pulposus progenitor cells from desired cells such as terminally differentiated cells or pluripotent or multipotent stem cells.

### Solution to Problem

The inventors first screened transcription factors specific to nucleus pulposus cells through a microarray assay and the like. Further, the inventors have found that a combination of specific transcription factors selected from the candidates, typically, a combination of Brachyury (T) with SRY-box6 (SOX6) and/or Forkhead Box Q1 (FOXQ1) serves as a set of potent transcription factors which should be called “nucleus pulposus cell master regulator transcription factors” that enable induction into an active nucleus pulposus cell phenotype not only by differentiation induction from stem cells such as MSCs but also transdifferentiation, for example, from terminally differentiated cells such as fibroblasts (PCT/JP2020/004449).

However, an active nucleus pulposus cell phenotype to be obtained by the aforementioned nucleus pulposus cell master regulator transcription factors is mature cells as nucleus pulposus cells, the expression levels (the positive rates in the cell population) of cell markers representing nucleus pulposus progenitor cells, i.e., Tie2 and GD2 are low, and the proportion of spherical colony-forming units (S-CFUs) is also low. For the production of cell preparations for regenerative medicine in the future, it would be more desirable to obtain a nucleus pulposus cell population that (i) has not lost the ability (phenotype) as progenitor cells, (ii) has a high ability to produce an extracellular matrix (ECM), and (iii) has a high ability to proliferate in vitro, enable large-scale batch production, and thus can improve marketability. From such a viewpoint, the aforementioned nucleus pulposus cell master regulator transcription factors have room for enhancement or improvement.

As a result of further studies to meet the aforementioned requirements, the inventors have found that differentiation of nucleus pulposus progenitor cells having a high ability to proliferate cells and capable of producing many active nucleus pulposus cell phenotypes (chondrogenic nucleus pulposus cells) from other nucleated cells can be induced by using a set of transcription factors in which MYC Proto-Oncogene, BHLH Transcription Factor (c-Myc, also known as CMYC or MYC, and hereinafter referred to as “cMyc” in the present description) is added to a combination of T with SOX6 and/or FOXQ1, that is, a combination of T, SOX6 and/or FOXQ1, and cMyc serves as nucleus pulposus progenitor cell master regulator transcription factors, thereby accomplishing the present invention.

That is, the present invention provides at least the following items.

[1] A nucleus pulposus progenitor cell inducer that is an agent comprising an effective amount of genes of transcription factors for induction of a nucleated cell other than nucleus pulposus progenitor cells into nucleus pulposus progenitor cells (hereinafter referred to as “nucleus pulposus progenitor cell master regulator transcription factors”) or products thereof (hereinafter referred to as “nucleus pulposus progenitor cell inducer”), wherein

the nucleus pulposus progenitor cell master regulator transcription factors comprise Brachyury (T) or a homolog thereof, at least one selected from the group consisting of SRY-box6 (SOX6) or a homolog thereof and Forkhead Box Q1 (FOXQ1) or a homolog thereof, and MYC Proto-Oncogene, BHLH Transcription Factor (cMyc) or a homolog thereof.

[2] The nucleus pulposus progenitor cell inducer according to item [1], wherein the nucleus pulposus progenitor cell master regulator transcription factors are in the form of genes inserted into an expression vector(s).

[3] A pharmaceutical composition for treating or preventing a spine-related disease in a vertebrate animal, comprising the nucleus pulposus progenitor cell inducer according to item [1] or [2].

[4] A method for producing an induced nucleus pulposus progenitor cell, comprising the steps of:

introducing the nucleus pulposus progenitor cell inducer according to item [1] or [2] in vitro into a nucleated cell other than a nucleus pulposus progenitor cell (hereinafter referred to as “introduction step”); and

performing induction into the nucleus pulposus progenitor cell through culturing the cell obtained by the introduction step (hereinafter referred to as “transcription factors-introduced cell”) (hereinafter referred to as “induction step”).

[5] The method for producing an induced nucleus pulposus progenitor cell according to item [4], further comprising a step of checking an expression status of at least one selected from the group consisting of Tie2, GD2, and CD24 in the cell during culture or after culture in the induction step.

[6] The method for producing an induced nucleus pulposus progenitor cell according to item [4] or [5], further comprising a step of checking whether the cell during culture or after culture in the induction step is capable of forming a colony-forming unit under colony-forming assay culture conditions.

[7] The method for producing an induced nucleus pulposus progenitor cell according to any one of items [4] to [6], further comprising a step of checking whether the cell during culture or after culture in the induction step is capable of differentiating into a nucleus pulposus cell.

[8] The method for producing an induced nucleus pulposus progenitor cells according to any one of items [4] to [7], wherein the induction step comprises culturing the transcription factors-introduced cell in a medium supplemented with basic fibroblast growth factor (bFGF or FGF2), epidermal growth factor (EGF), or both of them.

[9] The method for producing an induced nucleus pulposus progenitor cell according to any one of items [4] to [8], wherein the induction step comprises culturing the transcription factors-introduced cell under at least one condition selected from the group consisting of a hypoxic environment, an acidic environment, and a low glucose environment.

[10] The method for producing an induced nucleus pulposus progenitor cell according to any one of items [4] to [9], wherein the induction step is performed under colony-forming assay culture conditions.

[11] A transcription factors-introduced cell that is a cell comprising an effective amount of the nucleus pulposus progenitor cell master regulator transcription factors defined in item [1] or [2].

[12] An induced nucleus pulposus progenitor cell that is a cell having an active nucleus pulposus progenitor cell phenotype obtained through culturing the transcription factors-introduced cell according to item [11].

[13] The induced nucleus pulposus progenitor cell according to item [12], wherein the induced nucleus pulposus progenitor cell is expressing at least one selected from the group consisting of Tie2, GD2, and CD24.

[14] The induced nucleus pulposus progenitor cell according to item [12] or [13], wherein the induced nucleus pulposus progenitor cell is capable of differentiating into at least one mature cell phenotype selected from a nucleus pulposus cell phenotype and a notochord cell phenotype.

[15] A cell population comprising the transcription factors-introduced cell(s) according to item [11] and/or the induced nucleus pulposus progenitor cell(s) according to any one of items [12] to [14].

[16] A cell preparation for treating or preventing a spine-related disease in a vertebrate animal, comprising the induced nucleus pulposus progenitor cell(s) according to any one of items [12] to [14] or the cell population according to item [15].

[17] A method for treating or preventing a spine-related disease in a vertebrate animal (excluding human), comprising transplanting or administering the induced nucleus pulposus progenitor cell(s) according to any one of items [12] to [14], the cell population according to item [15], or the cell preparation according to item [16] in vivo so as to act on the intervertebral disc nucleus pulposus tissue.

[18] A method for treating or preventing a spine-related disease in a vertebrate animal (excluding human), comprising administering the nucleus pulposus progenitor cell inducer according to item [1] or [2] or the pharmaceutical composition according to item [3] in vivo so as to act on nucleus pulposus cells in an intervertebral disc.

[19] A method for screening a medicine or a method for treating or preventing a spine-related disease in a vertebrate animal, comprising a step of testing effectiveness and safety in a subject using the transcription factors-introduced cell(s) according to item [11], the induced nucleus pulposus progenitor cell(s) according to any one of items [12] to [14], or the cell population according to item [15].

[20] A method for obtaining an indicator associated with aging, degeneration, or disease state of an isolated nucleus pulposus cell population, comprising measuring the expression level of the nucleus pulposus progenitor cell master regulator transcription factors defined in item [1] or [2] in the nucleus pulposus cell population.

[21] A method for producing an induced nucleus pulposus cell, comprising a step of performing differentiation induction in vitro into an active nucleus pulposus cell or maturing the cell through culturing the induced nucleus pulposus progenitor cell according to any one of items [12] to [14].

[22] A kit comprising the nucleus pulposus progenitor cell inducer according to item [1] or [2].

In the present invention, the provision “in vitro” and “excluding human” is made only in view of industrial applicability, and the present invention can be performed “in vivo” and the application target of the present invention can be “including human” from a technical point of view. It is obvious for a person skilled in the art that, unless otherwise specified, a description on the present invention using a singular term (for example, a term with “a” or “an” in the case of English) and a description on the present invention using a plural term can be converted to each other. For example, a matter relating to the present invention described using a singular term is not only applicable to an embodiment in which a single object of the term is present, but also applicable to an embodiment in which a plurality of objects of the term are present, and the matter can be interpreted as applied to each object contained in a part of or all the plurality of objects. A matter relating to the present invention described using a plural term can be interpreted as applied generally to each object contained in a part or all of a plurality of objects of the term in an embodiment in which the plurality of objects are present.

### Advantageous Effects of Invention

The present invention enables cell transcription profiles to be directly changed, that is, cell transcription factor profiles to be induced into the same profiles as those of nucleus pulposus progenitor cells by specifically stimulating the master regulator of an active nucleus pulposus progenitor cell phenotype using a combination of specific transcription factors. This approach enables cells to be directly modified without the need for specific cell membrane receptors or related signaling proteins and therefore can be implemented relatively inexpensively with reliability (reproducibility).

Further, the nucleus pulposus progenitor cell master regulator transcription factors specified by the present invention are potent and thus expand the range of cells to which direct reprogramming can be applied. The present invention also enables differentiation induction of pluripotent or multipotent cell types (such as MSCs) into nucleus pulposus progenitor cells or transdifferentiation of mature differentiated cells, for example, terminally differentiated human dermal fibroblasts into nucleus pulposus progenitor cells. The nucleus pulposus progenitor cell master regulator transcription factors specified by the present invention allow nucleus pulposus progenitor cells with excellent proliferative capacity and producibility of spherical colony-forming units, and high expression levels of nucleus pulposus progenitor cell markers such as Tie2 and GD2 to be obtained. Further, nucleus pulposus cells (active nucleus pulposus cell phenotype) with excellent producibility of nucleus-related extracellular matrix such as proteoglycan and type II collagen can be obtained in a large amount at low cost through culturing the induced nucleus pulposus progenitor cells obtained by the present invention. The present invention can be further implemented by an embodiment of introducing the nucleus pulposus progenitor cell master regulator transcription factors into senescent cells or fibrous nucleus pulposus cells contained in an intervertebral disc or dedifferentiated nucleus pulposus cells during culture, to “redifferentiate” the cells into active nucleus pulposus progenitor cells.

Currently, effective treatment for chronic low back pain or the like is limited to symptomatic therapy, but application of the present invention enables mass supply of superior cell products for intervertebral disc regenerative medicine as off-the-shelf (OTS) cell preparations at low cost, thereby solving the problem of the number and quality of donors and the problem of production cost. The situation in which nucleus pulposus cells to be transplanted are collected from a tissue removed from affected areas of intervertebral disc degeneration such as scoliosis and intervertebral disc hernia or other organs, as before, can be overcome, and functional nucleus pulposus cells that are more healthy and closer to the original traits can be fabricated from various somatic cell types such as cells contained in the skin or adipose tissue. Further, nucleus pulposus progenitor cells to be fabricated by the present invention can also be autologous, and therefore the risk of using non-autologous cells can be avoided.

According to another aspect, induced nucleus pulposus progenitor cells to be obtained by the present invention can be fabricated from healthy individual donor cells and therefore can also be used for studying the health state of nucleus pulposus of young individuals under in-vitro conditions. Further, the present invention enables nucleus pulposus progenitor cells derived from patients presenting specific gene mutations that affect the spinal or general cell biology to be fabricated. That is, the present invention enables induced nucleus pulposus progenitor cells to be fabricated from cells contained in easy-to-access tissue sources such as the skin or adipose tissue of patients, and enables how gene mutations in the original cells affect the behavior of nucleus pulposus progenitor cells in vitro to be evaluated. Finally, use of induced nucleus pulposus progenitor cells to be obtained by the present invention in a personalized medical care strategy enables how medicine that becomes a treatment candidate and its dose affect induced nucleus pulposus progenitor cell populations specific to the patient to be determined, thereby revealing patient-specific potential negative effects before actual administration to the patient, which can be useful for knowing an appropriate treatment method.

### BRIEF DESCRIPTION OF DRAWINGS

FIG. 1[A] describes changes in expression of Tie2, GD2, and CD24 in a simplified hierarchical differentiation process from nucleus pulposus progenitor cells into nucleus pulposus cells, along the differentiation axis (Sakai D, Nakamura Y, Nakai T, et al. Exhaustion of nucleus pulposus progenitor cells with ageing and de-generation of the intervertebral disc, Nat Commun. 2012; 3: 1264./Sakai D, Schol J, Bach F C, et al. Successful fishing for nucleus pulposus progenitor cells of the intervertebral disc across species. JOR Spine. 2018; 1 (2): e1018.). FIGS. 1[B] and 1[C] show the results of sorted monolayer-cultured nucleus pulposus cell populations by Tie2 expression (Tie2⁺), G-D2 expression (GD2⁺), or CD24 expression (CD24⁺), or the results of sorted nucleus pulposus cell populations cultured in methylcellulose in colony-forming assay by CD24 expression (CD24⁺,3D). The expression levels of the transcription factors in nucleus pulposus cells are compared with the expression profiles of iPS cells (iPSC), lung cells (Lung), neural progenitor cells (NPC), and fibroblasts (Fibro). FIG. 1[B] shows the top half of the highly-expressed transcription factors, and FIG. 1[C] shows the bottom half of the hits.

FIG. 2 includes optical micrographs (upper row) and fluorescence micrographs (lower row) of cells obtained by transducing specific combinations of GFP (SHAM), Brachyury (T), FOXQ1 (F), SOX6 (S), and cMyc (C) into mesenchymal stromal cells (MSCs) to be overexpressed, followed by culture for one week. The morphological changes clearly show the differences between the culture conditions. TF, TF+C, TSF, and TSF+C show morphologies similar to those of the nucleus pulposus cell phenotype. The “t0” represents MSCs before transduction. The “NC” (negative control) represents MSCs cultured under the same conditions except that transduction was not performed (the same applies to the following figures). The “SHAM” represents MSCs in which only GFP was transduced and overexpressed as a control (the same applies to the following figures). Except for the SHAM control, GFP expression serves as a reporter of T expression.

FIG. 3 includes gene expression profiles of cells obtained by transducing a combination of Brachyury (T), SOX6 (S), and FOXQ1 (F) with addition of cMyc (+C) and without addition of cMyc into mesenchymal stromal cells (MSCs) to be over-expressed, followed by culture for 2 weeks. The expression profiles are shown as values relative to the housekeeping gene GAPDH and the negative control (NC: non-transduced MSCs). The SHAM control represents MSCs transduced to overexpress GFP. The expression levels of [A] COL2A1, [B] CD24, [C] ACAN, [D] KRT8, [E] PITX1, and [F] PAX1 are measured. [G] is a heat map showing combinations of average expression levels of the specific genes, indicating the impact of each combination of transduced genes on the overall expression levels. In general, the combination of TF and TSF showed high effectiveness for expression of NP markers, and addition of C could sustain or enhance their expression.

FIG. 4 includes gene expression profiles of cells obtained by transducing combination of Brachyury (T), SOX6 (S), and FOXQ1 (F) with addition of cMyc (+C) and without addition of cMyc into mesenchymal stromal cells (MSCs) to be over-expressed, followed by culture for about 3 weeks. It was suggested that the addition of cMyc improved the cell proliferation rate.

FIG. 5[A] includes optical micrographs and fluorescence micrographs of cells obtained by transducing a combination of Brachyury, FOXQ1, and cMyc (TF+C) into MSCs, followed by culture for one week for colony-forming assay (according to Sakai et al. JOR Spine. 2018; 1 (2): e1018.), The TF+C-transduced MSCs exhibit huge spherical colony-forming units producing GFP (that is, expressing T). FIG. 5[B] shows the results of measuring the number of spherical colony-forming units (CFUs-S) per 1000 cells in MSCs transduced with the combination of Brachyury (T), SOX6 (S), FOXQ1 (F), and cMyc (C), followed by culture for 10 weeks.

FIG. 6 shows the results of measuring the proportion of GFP (which serves as a reporter of T expression except for SHAM control)—positive cells (GFP-positive rate) in MSCs transduced with the specific combination of Brachyury (T), SOX6 (S), FOXQ1 (F), and cMyc (C), by flow cytometry at time point 1 (before transduction: t=0), time point 2 (after culture for 1 to 2 weeks from time point 1), and time point 3 (after culture for 1 to 2 weeks from time point 2).

FIG. 7 shows the results of measuring [A] Tie2-positive rate, [B] GD2-positive rate, and [C] CD24-positive rate for each of GFP (which serves as a reporter of T expression except for SHAM control)—positive cell fractions (+) and—negative cell fractions (−) in MSCs transduced with the specific combination of Brachyury (T), SOX6 (S), FOXQ1 (F), and cMyc (C), by flow cytometry at time point 2 (after culture for 1 to 2 weeks from transduction) and time point 3 (after culture for 1 to 2 weeks from time point 2). [D] is a heat map showing an overview of the results of analyzing the positive rate of each nucleus pulposus progenitor cell marker by flow cytometry.

FIG. 8 includes observation images of MSCs transduced with the combination of Brachyury (T), SOX6 (S), FOXQ1 (F), and cMyc (C) (TSF+C) or the combination of T, F, and C (TF+C) or SHAM MSCs seeded on fragments of human nucleus pulposus tissues entirely treated with radiation and tested for migration, proliferation, and approximate presence of GFP protein 1 to 2 weeks later, with a fluorescence microscope (Olympus IX70, Olympus Corporation). For SHAM control, a uniformly distributed cell population with large fibroblast-like characteristics was observed. Meanwhile, for TSF+C and TF+C, GFP-positive cells were scattered on the outer layer of tissue fragments one week after seeding, but then changed to form a large, dense cell population mass, which suggests that the first-seeded cells have a proliferative stem cell-like phenotype.

FIG. 9 shows [A] the number and [B] viability (percentage with respect to the total number of cells) of MSCs transduced with the combination of Brachyury (T), SOX6 (5), FOXQ1 (F), and cMyc (C) (TSF+C), the combination of T, F, and C (TF+C), or C alone (C), or SHAM MSCs and collected after whole tissue culture for 2 weeks. In flow cytometry, dead cells were eliminated by PI (propidium iodide) staining.

FIG. 10 shows the results of measuring the Tie2-positive rate, the GD2-positive rate, and the CD24-positive rate for each of GFP (which serves as a reporter of T expression except for SHAM control)—positive cell fractions (+) and—negative cell fractions (−) in MSCs transduced with the combination of Brachyury (T), SOX6 (S), FOXQ1 (F), and cMyc (C) (TSF+C), the combination of T, F, and C (TF+C), or C alone (C), by flow cytometry after whole tissue culture for 2 weeks.

FIG. 11 includes optical micrographs (magnification 10×, each scale bar represents 250 μm) of induced nucleus pulposus cells obtained by transducing (A) fibroblasts and (B) MSCs with the master regulator transcription factors (four combinations of T, SOX6, and FOXQ1), followed by one-week culture, (A) Induced nucleus pulposus cells fabricated from human fibroblasts were contrasted with SHAM control (transduced with green fluorescent protein (GFP) instead of the master regulator transcription factors). (B) Likewise, induced nucleus pulposus cells fabricated from human bone marrow-derived MSCs were contrasted with SHAM control.

FIG. 12 includes optical micrographs (magnification 10× and 20×) of vacuoles observed within induced nucleus pulposus cells (in the protoplasm) one to two weeks after transduction. Human newborn dermal fibroblasts were transduced with three combinations of T, SOX6, and FOXQ1, and the SHAM control was transduced with GFP, instead. The black arrows point to the vacuoles within the cells.

FIG. 13 shows the results of examining the mRNA expression levels of nucleus pulposus cell markers (aggrecan (ACAN), type II collagen (COL2), type I collagen (COL1), CD24, keratin 18 (KRT18), and keratin 8 (KRT8)) by different combinations of master regulator transcription factors in induced nucleus pulposus cells derived from fibroblasts. In each graph, human dermal fibroblasts derived from three different donors were transduced with different combinations of T, SOX6 (S), and FOXQ1 (F), cultured for one week, and contrasted with the respective SHAM controls transduced with GFP. The mRNA expression level was calculated as a relative value to the GAPDH expression level, and a difference from the expression level of the SHAM control (relative value) was determined for each donor. The numerical value represents an average±standard error (SEM). For the statistical analysis, two-way analysis of variance (no matching) and Tukey's multiple comparison test were used, and p<0.05 was determined to be a significant difference (* p≤0.05, ** p≤0.01, *** p≤0.005, **** p≤0.001).

FIG. 14 includes optical micrographs (each scale bar represents 25 μm) showing the histological overview of induced nucleus pulposus cells in chondrogenic pellets. Hematoxylin/eosin and safranin-O/fast green stained sections of pellet cultures of fibroblasts or MSCs transduced with different combinations of brachyury (T), SOX6 (S), PITX1 (P), and FOXQ1 (F) were contrasted with the respective SHAM controls transduced with GFP. Fibroblasts transduced with a combination of TS and TSP, and MSCs transduced with TS show strong cell vacuolation bye hematoxylin/eosin staining. Meanwhile, the combination of TS and TSF in induced nucleus pulposus cells derived from both the fibroblasts and the MSCs showed a proteoglycan region or uniform deposition by red safranin-O staining.

### DESCRIPTION OF EMBODIMENTS

### Nucleus Pulposus Progenitor Cell Inducer (Nucleus Pulposus Progenitor Cell Master Regulator Transcription Factors

The nucleus pulposus progenitor cell inducer of the present invention is an agent containing a gene of transcription factors (nucleus pulposus progenitor cell master regulator transcription factors) or a product thereof in an effective amount for induction of cells into active nucleus pulposus progenitor cells. in the present invention, “inducing cells into nucleus pulposus progenitor cells” includes embodiments such as (i) maintaining “transdifferentiation” into nucleus pulposus progenitor cells from terminally differentiated cells other than nucleus pulposus progenitor cells or cells committed to differentiation into cells other than nucleus pulposus progenitor cells and inducing the cells into nucleus pulposus progenitor cells (which may be referred to as “first embodiment” in this description), (ii) differentiation induction into nucleus pulposus progenitor cells from stem cells or the like capable of differentiating into nucleus pulposus progenitor cells and other cells (having pluripotency or multipotency) (which may be referred to as “second embodiment” in this description), and (iii) reactivating dedifferentiated or otherwise compromised nucleus pulposus progenitor cells into nucleus pulposus progenitor cells (which may be referred to as “third embodiment” in this description). The nucleus pulposus progenitor cell inducer of the present invention can be used in any form of the first, second, and third embodiments, and the term “nucleus pulposus progenitor cell inducer” is used as a general term, but it is also possible to use the term “transdifferentiation agent” particularly for use in the first embodiment, the term “differentiation inducer” particularly for use in the second embodiment, and the term “reactivation agent” particularly for use in the third embodiment.

The combination of nucleus pulposus progenitor cell master regulator transcription factors as the subject of the present invention, that is, the combination of master regulator transcription factors important for maintaining the phenotype of nucleus pulposus progenitor cells includes: (1) Brachyury (T) or a homolog thereof, (2) at least one selected from the group consisting of SRI-box 6 (SOX6) or its homolog and Forkhead Box Q1 (FOXQ1) or its homolog (at least one selected from the group consisting of SRI-box 6 (SOX6) and Forkhead Box Q1 (FOXQ1) or a homolog(ue) thereof), and (3) MYC Proto-Oncogene, BHLH Transcription Factor (cMyc) or a homolog thereof. The aforementioned transcription factor (2) preferably contains at least FOXQ1 or its homolog, more preferably both FOXQ1 or a homolog thereof and SOX6 or a homolog thereof. The terms “T”, TSOX6”, “FOXQ1” and “cMyc” (and other terms representing other transcription factors described herein) do not limit whether the transcription factors are genes (nucleic acids) or products thereof (proteins), and the terms may be interpreted as being genes (nucleic acids) or products thereof (proteins) according to the context.

The homologue) of each nucleus pulposus progenitor cell master regulator transcription factor is known to those skilled in the art and can be searched by databases such as DNA data bank of Japan (DDBJ), NCBI GenBank, and EMBL. Which (combination of) nucleus pulposus progenitor cell master regulator transcription factors are to be selected can be appropriately adjusted corresponding to whether the nucleus pulposus progenitor cell inducer of the present invention is to be used as a transdifferentiation agent (for terminally differentiated cells or the like other than nucleus pulposus progenitor cells), as a differentiation inducer (for stem cells or the like capable of differentiating into nucleus pulposus progenitor cells and other cells), or as a reactivation agent (for dedifferentiated or damaged nucleus pulposus progenitor cells).

In the present invention, the “nucleus pulposus progenitor cells” refer to cells having at least one, preferably, both traits of (i) being positive for the expression of at least one selected from the group consisting of Tie2, GD2, and CD24, and (ii) having the ability to form spherical colony-forming units under colony-forming assay culture conditions. Not all cells belonging to the cell population to which the present invention is applied need to have the same traits uniformly, and cells having the aforementioned trait (i), preferably, further having the aforementioned trait (ii) may he present at least as a part of the cell population. In the present invention, a cell population comparatively rich in nucleus pulposus progenitor cells having the aforementioned trait (i), preferably, further having the aforementioned trait (ii) can be prepared. The aforementioned nucleus pulposus progenitor cells are capable of differentiating into nucleus pulposus cells (active nucleus pulposus cell phenotype) but also are capable of differentiating into multi-series cell phenotypes such as chondrocytes, adipocytes, osteocytes, and nerve cells.

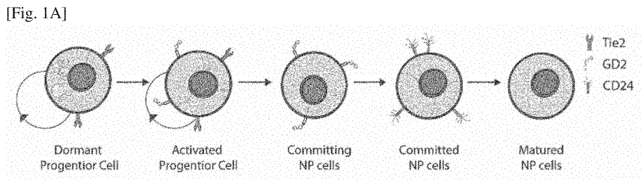

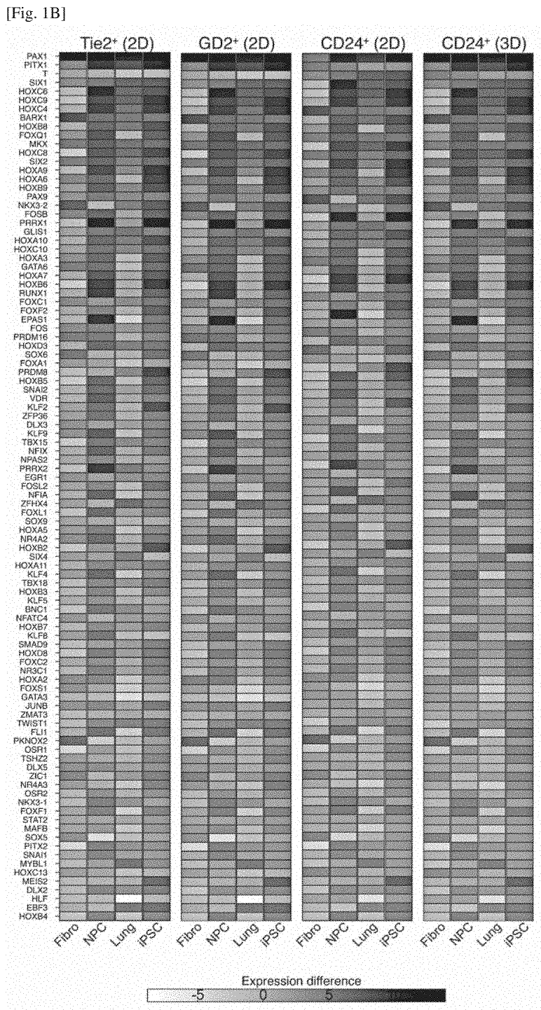

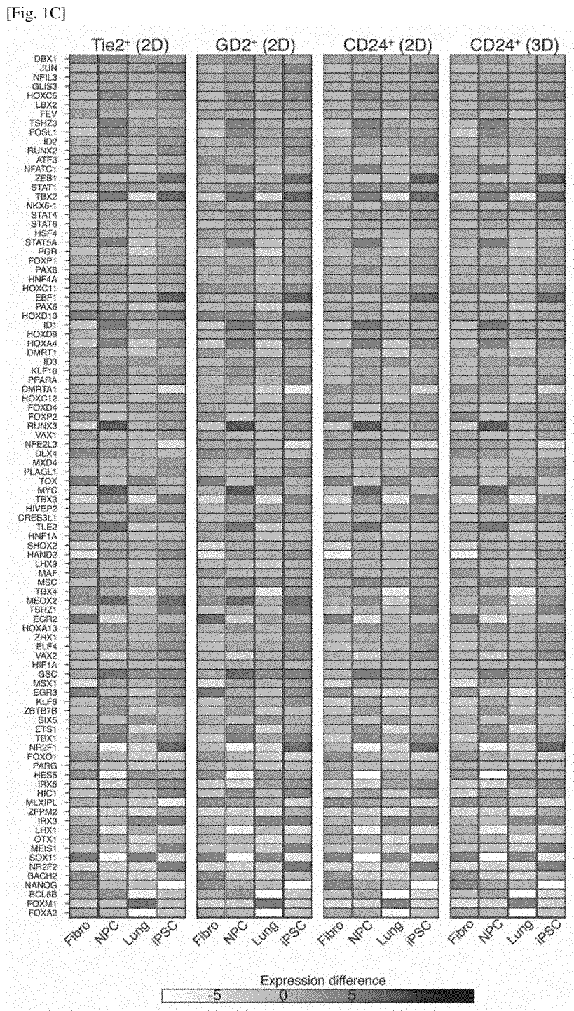

In a preferable embodiment of the present invention, the “nucleus pulposus progenitor cells” of the present invention are Tie2-positive, GD2-positive, and CD24-negative (Tie2+/CD2+/CD24−) cells, that is, cells of “active nucleus pulposus progenitor cell phenotype” (“Activated Progenitor Cells” in FIG. 1). However, the “nucleus pulposus progenitor cells” of the present invention may refer to Tie2-positive, GD2-negative, and CD24-negative (Tie2+/GD2−/CD24−) cells, that is, cells of dormant nucleus pulposus progenitor cell phenotype (“Dormant Progenitor Cells” in FIG. 1); Tie2-negative, GD2-positive, and CD24-negative (Tie2−/CD2+/CD24−) cells (“Committing NP cells” in FIG. 1); Tie2-negative, GD2-negative, and CD24-positive (Tie2−/CD2−/CD24+) cells (“Committed NP cells” in FIG. 1), in other words, the nucleus pulposus progenitor cells may be cells other than Tie2-negative, GD2-negative, and CD24-negative (Tie2−/CD2−/CD24−) cells (“Matured NP cells” in FIG. 1), in a broad sense.

In the present invention, the “nucleus pulposus cells” (active nucleus pulposus cell phenotype) obtained by further differentiation induction of “nucleus pulposus progenitor cells” mean to have one or more traits (phenotypes) selected from (i) producing a large amount of nucleus pulposus-related matrix proteins such as proteoglycans (for example, aggrecan) and type II collagen or expressing at least one of cell markers specific to nucleus pulposus cells, such as CD24, KRT8, and KRT18, (ii) being capable of coping with hypoglycemia, acidity, hypoxia, and/or hyperosmolarity conditions imitating a healthy or moderately degenerated intervertebral disc, and (iii) having the same morphological characteristics as nucleus pulposus cells, such as cytoskeletal deposition and comparatively large intercellular vacuoles, preferably, to have traits of a plurality of groups (i), (ii), or (iii). Further, not all the cells belonging to the cell population necessarily have the same traits uniformly, and trait (iii) above may be seen only in a part of the cell population, for example.

In another embodiment of the present invention, the nucleus pulposus cell master regulator transcription factors may contain (1) T or a homolog thereof and (3) cMyc or a homolog thereof (that is, not containing the aforementioned factor (2)), or may contain (2) at least one selected from the group consisting of SRY-box 6 (SOX6) or its homolog and Forkhead Box Q1 (FOXQ1) or its homolog (at least one selected from the group consisting of SOX6 and FOXQ1 or a homolog(ue) thereof) and (3) cMyc or a homolog thereof (that is, not containing the aforementioned factor (1)).

The nucleus pulposus progenitor cell master regulator transcription factors of the present invention may further contain transcription factors other than the aforementioned predetermined transcription factors, as required, without impairing the actions and effects of the present invention. In one embodiment of the present invention, the nucleus pulposus progenitor cell master regulator transcription factors can further contain at least one selected from the group consisting of PITX1 or its homolog and PAX1 or its homolog (PITX1 and PAX1 or a homolog(ue) thereof).

In one embodiment of the present invention, the nucleus pulposus progenitor cell master regulator transcription factors can contain at least one selected from the group consisting of HIF3α or its homolog, SOX9 or its homolog, RUNX1 or its homolog, HIF1α or its homolog, and FOXA2 or its homolog (HIF3α, SOX9, RUNX1, HIF1α, and FOXA2 or a homolog(ue) thereof).

The nucleus pulposus progenitor cell master regulator transcription factors of the present invention may be introduced into target cells in the form of a gene (nucleic acid) or in the form of a protein that is a product of the gene. A variety of means for introducing the gene (nucleic acid) or protein into the target cells are known to those skilled in the art, and suitable means and conditions corresponding to the mean can be used in the present invention. The gene (nucleic acid) of the nucleus pulposus progenitor cell master regulator transcription factors may be, for example, in the form of DNA such as plasmid or in the form of RNA such as mRNA, and the gene can be introduced into the target cells in each form, for example, by transfection using a composite with a liposome, lipid particles, a polymer, or the like; electroporation; or a viral vector using retrovirus, lentivirus, adeno-associated virus, adenovirus, Sendai virus, or the like. Further, a protein of the nucleus pulposus progenitor cell master regulator transcription factors can be introduced into the target cells, for example, by coupling cell penetrating peptides with the protein.

According to one embodiment of the present invention, the nucleus pulposus progenitor cell master regulator transcription factors are introduced into the target cells in the form of an expression vector (such as a viral vector plasmid and an expression plasmid) into which a gene encoding at least one of the nucleus pulposus progenitor cell master regulator transcription factors has been inserted. The gene on the vector is expressed within the target cells, thereby producing a protein of the nucleus pulposus progenitor cell master regulator transcription factor, as a result of which, gene expression specific to an active nucleus pulposus progenitor cell phenotype such as Tie2 and GD2 is directly or indirectly induced, The vector may be double-stranded or single-stranded and may be DNA or RNA. The vector may be an embodiment of existing in the nucleus or cytoplasm either temporarily or continuously while being replicated, or may be an embodiment of permanently existing in the genomic DNA while being incorporated therein. In the case where a plurality of nucleus pulposus progenitor cell master regulator transcription factors are expressed, one of the nucleus pulposus progenitor cell master regulator transcription factors may be expressed by one expression vector (using a plurality of such expression vectors in combination), or the plurality of nucleus pulposus progenitor cell master regulator transcription factors may be expressed by one expression vector.

For example, plasmid DNAs containing genes encoding T, SOX6, FOXQ1, and cMyc may be introduced into human mesenchymal stem cells or mesenchymal stromal cells (MSCs) derived from bone marrow or the like or human fibroblasts by transfection using lentivirus, and the factors can be expressed by a 35M cauliflower mosaic virus promoter (pCMV) disposed in the upstream of the genes, This causes overexpression of the T, SOX6, FOXQ1, and cMyc genes in the human mesenchymal stem cells or human fibroblasts having the four nucleus pulposus progenitor cell master regulator transcription factors introduced thereinto, so that the cells can be induced into nucleus pulposus progenitor cells. The induced nucleus pulposus progenitor cells thus obtained are characterized by expression and production of nucleus pulposus progenitor cell-specific markers such as Tie2, GD2, and CD24.

According to one embodiment of the present invention, the nucleus pulposus progenitor cell master regulator transcription factors of the present invention function as markers of an activated nucleus pulposus progenitor cell phenotype. The nucleus pulposus progenitor cell master regulator transcription factors are rarely expressed in the cell population mainly composed of nucleus pulposus cells that have aged and differentiated into a fibrous cell type, such as a degenerated intervertebral disc. In contrast, the expression levels in the cell population mainly composed of healthy nucleus pulposus progenitor cells are comparatively high. Therefore, the expression levels (positive rates) of the nucleus pulposus progenitor cell master regulator transcription factors in a cell population that contains isolated and cultured nucleus pulposus progenitor cells or a cell population that contains nucleus pulposus progenitor cells collected from a tissue such as a nucleus pulposus and an intervertebral disc, mature nucleus pulposus cells, or the like are measured, thereby enabling indices of the phenotype of nucleus pulposus progenitor cells, the cell activity, and the health of the cell population or the tissue to be obtained as indices associated with the aging, degenerating or disease state of the cell population containing nucleus pulposus progenitor cells and the like.

### Method for Producing Induced Nucleus Pulposus Progenitor Cells

The method for producing induced nucleus pulposus progenitor cells of the present invention includes at least the steps of introducing the nucleus pulposus progenitor cell inducer of the present invention (an effective amount of a gene of nucleus pulposus progenitor cell master regulator transcription factors or a product thereof) into a cell in vitro (introduction step); and performing induction into nucleus pulposus progenitor cells through culturing the cell obtained by the introduction step (induction step). The method for producing induced nucleus pulposus progenitor cells of the present invention preferably further includes at least one of a step of checking the expression status of at least one selected from the group consisting of Tie2, GD2, and CD24 in the cells during culture or after culture in the induction step (checking step 1), a step of checking whether the cells during culture or after culture in the induction step are capable of forming colony-forming units under colony-forming assay culture conditions (checking step 2), and a step of checking whether the cells during culture or after culture in the induction step are capable of differentiating into nucleus pulposus cells (checking step 3).

### Introduction Step

The introduction step is a step of introducing the nucleus pulposus progenitor cell inducer of the present invention (an effective amount of a gene of nucleus pulposus progenitor cell master regulator transcription factors or a product thereof) into cells.

The cells as targets for introduction of the nucleus pulposus progenitor cell inducer (herein referred to as “introduction target cells”) are not specifically limited, as long as they are nucleated cells other than nucleus pulposus progenitor cells, preferably, somatic cells (cells other than germ cells), and include various cell types. The introduction target cells may be an established cell line or may be primary cultured cells (autologous cells or allogeneic cells) collected from an individual or their passage cells.

In the first embodiment of the present invention, the introduction target cells are various somatic cells that have terminally differentiated into cells other than nucleus pulposus progenitor cells or cells that have already been committed to differentiation into cells other than nucleus pulposus progenitor cells (such as progenitor cells of a specific cell lineage other than nucleus pulposus progenitor cells) and are, for example, skin tissue cells that are relatively easy to obtain and culture, typically preferably, fibroblasts, but terminally differentiated (mature) nucleus pulposus cells can also be used.

In the second embodiment of the present invention, the introduction target cells are stem cells such as embryonic stem cells (ES cells) and induced pluripotent stem cells (iPS cells) possessing pluripotency, various stem cells possessing multipotency into nucleus pulposus progenitor cells and other cells, or other cells that have not been committed to differentiation into nucleus pulposus progenitor cells. The introduction target cells in the second embodiment of the present invention are, for example, preferably mesenchymal stromal cells or mesenchymal stem cells (MSCs), which are adult stem cells that can be collected from tissues such as fat, umbilical cord, synovium, and bone marrow, but a dormant nucleus pulposus progenitor cell phenotype can also be used.

The introduction target cells are generally derived from vertebrate animals, typically, mammals and may be derived from humans or non-human mammals. Examples of mammals can include mice, rats, dogs, cats, sheep, and bovines, in addition to humans. In the case where the introduction target somatic cells are primary cultured cells collected from an individual or their passage cells, the individual may be an individual into which induced nucleus pulposus progenitor cells are to be transplanted (such as patients of intervertebral disc disorders) or may be an individual different from the aforementioned individual (a healthy individual or a donor).

The introduction target cells may be derived from a vertebrate animal with genetic variation. For example, nucleus pulposus progenitor cell master regulator transcription factors are introduced into skin fibroblasts derived from a target (a human or a non-human vertebrate) with genetic variation to produce induced nucleus pulposus progenitor cells, thereby enabling analysis of the influence of genetic variation (the role of the gene) on the behavior of nucleus pulposus cells, intervertebral disc development, and other phenomena.

The nucleus pulposus progenitor cell inducer used in the introduction step may be used in an effective (necessary) amount (i) for maintaining and inducing transdifferentiation, in the case where the introduction target cells are terminally differentiated into cells other than nucleus pulposus progenitor cells or the like, from the cells into nucleus pulposus progenitor cells (preferably, an active nucleus pulposus progenitor cell phenotype), or an effective (necessary) amount (ii) for differentiation induction, in the case where the introduction target cells are undifferentiated cells or the like, from the cells into nucleus pulposus progenitor cells (preferably, an active nucleus pulposus progenitor cell phenotype). The amount to be used as the transdifferentiation agent (i) and the amount to be used as the differentiation inducer (ii) each vary depending on the embodiment of the present invention, such as the type of introduction target cells, the types of nucleus pulposus progenitor cell master regulator transcription factors and selection of whether they are introduced into the cells in the form of a gene (such as expression plasmid and mRNA) or in the form of a protein and further the means (such as a viral vector, electroporation, microinjection, lipofection, coupling of cell penetrating peptides, and other transfection reagents) for introducing the factors into cells, and the cell culture conditions. The numerical range thereof cannot be generally determined. Those skilled in the art would be able to adjust and set the amount to be used, for example, using the ratio of the number of induced nucleus pulposus progenitor cells with respect to the total number of cells as an indicator, so that an expected purpose can be achieved. All types of the genes of nucleus pulposus progenitor cell master regulator transcription factors or products thereof may be introduced in multiple steps in a desired order but are preferably introduced in one step.

As an indicator, it is conceivable to set the number of viral vectors per cell to a suitable range according to the concept of the multiplicity of viral infection (MOI), when nucleus pulposus progenitor cell master regulator transcription factors are introduced into the introduction target cells in the form of genes using viral vectors. As an example, it is conceivable to use a viral vector solution so that about 8 viral vectors are introduced into each cell (equivalent to MOI=8). Since the number of “about 8” above is mentioned as an example, a larger or smaller number may be employed, and the number can be appropriately set and adjusted by those skilled in the art. Likewise, also in the embodiment in which genes of nucleus pulposus progenitor cell master regulator transcription factors are introduced into the cells in a form other than viral vectors, such adjustment can be performed so that an appropriate number of genes of nucleus pulposus progenitor cell master regulator transcription factors are introduced into each introduction target cell.

Accordingly, the nucleus pulposus progenitor cell inducer of the present invention can be configured as a solution containing genes of nucleus pulposus progenitor cell master regulator transcription factors prepared so as to have an appropriate “multiplicity of infection” (such as expression vectors introduced into each cell) or a kit therefor, corresponding to the type of introduction target cells, the number of cells, and other culture conditions.

The introduction step may be performed under conditions suitable for introducing an effective amount of a gene of a nucleus pulposus progenitor cell master regulator transcription factor or a product thereof into introduction target cells, and culturing the cells after introduction. The culture conditions such as components in the culture medium used therefor (such as the basal culture medium, growth factors, other additive components, vectors, and transfection reagents), the culture period, and the atmosphere can be appropriately set by those skilled in the art.

### Induction Step

The induction step is a step of culturing the transcription factors-introduced cell obtained in the introduction step with the nucleus pulposus progenitor cell inducer of the present invention (an effective amount of the gene of a nucleus pulposus progenitor cell master regulator transcription factor or a product thereof) introduced and inducing the cells into nucleus pulposus progenitor cells (preferably, an active nucleus pulposus cell phenotype).

The induction step may be performed under conditions suitable for culturing until the cells having the nucleus pulposus progenitor cell master regulator transcription factors introduced thereinto become nucleus pulposus progenitor cells (preferably, an active nucleus pulposus progenitor cell phenotype). The culture conditions such as components in the culture medium used therefor (such as the basal culture medium, growth factors, and other additive components), the culture period, and the atmosphere can be appropriately set by those skilled in the art. Further, the culture in the induction step may be two-dimensional culture (for example, monolayer culture) or three-dimensional culture (for example, 3D pellet culture, whole tissue culture (method of culturing nucleus pulposus progenitor cells while they are held in the niche of the inter-vertebral disc nucleus pulposus tissue instead of isolating and culturing nucleus pulposus progenitor cells from the intervertebral disc nucleus pulposus tissue), and culture in methylcellulose medium).

In one embodiment of the present invention, the medium (cell culture) in the induction step can be supplemented with basic fibroblast growth factor (which may be referred to as bFGF or FGF2), epidermal growth factor (EGF), or both of them. The concentrations of bFGF (FGF2) and EGF in the medium can be each appropriately adjusted in consideration of the action of induction into the active nucleus pulposus progenitor cell phenotype. The concentration of bFGF (FGF2) is generally 0.1 to 100,000 ng/mL, preferably 10 to 100 ng/mL, and the concentration of EGF is generally 0.1 to 100,000 preferably 10 to 100 ng/mL.

In one embodiment of the present invention, the medium in the induction step can contain at least one selected from the group consisting of transforming growth factor β1 (TGFβ1), transforming growth factor β2 (TGFβ), and transforming growth factor β3 (TGFβ3), and/or at least one selected from the group consisting of growth differentiation factor 5 (GDF5) and growth differentiation factor 6 (GDF6), for example, in the case where nucleus pulposus progenitor cells are obtained in the induction step, and then nucleus pulposus cells (preferably, an active nucleus pulposus cell phenotype) are further obtained from the nucleus pulposus progenitor cells. The concentration of at least one selected from the group consisting of TGFβ1, TGFβ2, and TGFβ3 in the medium can be appropriately adjusted but is generally 1 to 10,000 ng/mL, preferably 10 to 100 ng/mL, for example, about 10 ng/mL. The concentration of at least one selected from the group consisting of GDF5 and GDF6 in the medium can be appropriately adjusted but is generally 1 to 100,000 ng/mL, preferably 10 to 500 ng/mL, for example, about 100 ng/mL.

Further, the medium in the induction step can contain at least one selected from the group consisting of dexamethasone, L-ascorbic acid, fetal bovine serum (FBS), and ITS-X (Insulin-Transferrin-Selenium-Ethanolamine), preferably all of them, in an appropriate amount. The concentration of dexamethasone in the medium is generally 0.1 to 1,000 ng/mL, preferably 4 to 500 ng/mL, for example, about 10 ng/mL. The concentration of L-ascorbic acid in the medium is generally 1 to 1,000 μM, preferably 5 to 500 μM, for example, about 50 μM. The concentration of FBS in the medium is generally 0.5 to 40%. The concentration of ITS-X in the medium is generally 0.1 to 5%.

According to one embodiment of the present invention, the induction step can include culturing the cells under at least one condition selected from the group consisting of a hypoxic environment, an acidic environment, and a low glucose environment, more preferably under all these conditions. Induced nucleus pulposus progenitor cells which are viable in a hypoglycemic, acidic, and hypoxic (and hyperosmolar state) environment that is the environment in a healthy or moderately degenerated intervertebral disc can be fabricated and recovered by culturing cells having nucleus pulposus progenitor cell master regulator transcription factors introduced thereinto in such an environment. The hypoxic environment generally refers to an environment in which the oxygen concentration in the atmosphere of the culture medium is 1 to 10%, preferably 2 to 5%, for example, about 2%. In the case where the induction step is not performed under hypoxic conditions, the oxygen concentration in the atmosphere of the medium can be, for example, about 21%. The acidic environment generally refers to an environment in which the pH of the culture medium at room temperature (for example, 25° C.) is in the range of 6.5 to 7.4, for example, about 6.8. The low glucose environment generally refers to an environment in which the glucose concentration in the culture medium is 4.5 g/L or less, for example, about 1 g/L. The culture period under such an environment can be appropriately adjusted but is generally 2 to 90 days (3 months), for example, 14 days (two weeks), For example, it is preferable to culture cells having with nucleus pulposus progenitor cell master regulator transcription factors introduced thereinto under a hypoxic environment with an oxygen concentration of 2% for two weeks.

### Checking Step 1

In checking step 1, the expression status of a gene or a protein that characterizes induced nucleus pulposus progenitor cells, that is, a nucleus pulposus progenitor cell marker in the cells during culture or after culture in the induction step is checked. As such a nucleus pulposus progenitor cell marker, either a marker that is positive in an active nucleus pulposus progenitor cell phenotype (positive marker) or a marker that is negative therein (negative marker) can be used. The nucleus pulposus progenitor cell marker may be a gene or a protein but is, for example, preferably a protein serving as a cell surface marker (to check the expression status of the protein). The marker that is a gene and/or a protein in the checking target cells can be checked quantitatively or qualitatively, for example, by a general approach such as real time polymerase chain reaction (real time PCR), immunohistochemistry staining (IHC), Western blotting, and flow cytometry. Whether the expression of each marker is positive or negative and further whether the marker in the checking target cells has a predetermined expression profile can be determined based on the results.

More specifically, checking step 1 is a step of checking the expression status of at least one selected from the group consisting of Tie2, GD2, and CD24 in the cells during culture or after culture in the induction step. The “expression of at least one selected from the group consisting of Tie2, GD2, and CD24” serves as a cell surface marker that characterizes nucleus pulposus progenitor cells (and nucleus pulposus cells) (see FIG. 1). The expression of the aforementioned three types may be for genes or proteins but is preferably for proteins. The expression status of the aforementioned three types of cell surface markers can be checked quantitatively or qualitatively, for example, by a general approach such as real time polymerase chain reaction (real time PCR), immunohistological staining (IHC), Western blotting, and flow cytometry. Whether the expression is positive or negative can be determined based on the results.

For example, the fact that the cells during culture or after culture in the induction step are positive for Tie2, positive for GD2, and negative for CD24 (Tie2+/GD2+/CD24−) indicates that the cells have an active nucleus pulposus progenitor cell phenotype. The cell population containing a certain number of cells having such an expression profile can be used as one of the indices for determining that a certain number of nucleus pulposus progenitor cells (active nucleus pulposus progenitor cell phenotype) have been obtained by the induction step. Likewise, the fact that the cells are positive for Tie2, negative for GD2, and negative for CD24 (Tie2+/GD2−/CD24−) indicates that the cells are of dormant nucleus pulposus progenitor cell phenotype, the fact that the cells are negative for Tie2, positive for GD2, and negative/positive for CD24 (Tie2−/GD2+/CD24−/+) indicates that the cells are committed to nucleus pulposus cells (differentiation into nucleus pulposus cells has been directed), the fact that the cells are negative for Tie2, negative for GD2, and positive for CD24 (Tie2−/GD2−/CD24+) indicates that the cells are committed to nucleus pulposus cells (differentiation into nucleus pulposus cells has been determined), and the fact that the cells are negative for Tie2, negative for GD2, and negative for CD24 (Tie2−/GD2−/CD24−) and further a nucleus pulposus cell marker, which will be described below, has a predetermined expression profile indicates that the cells have become mature nucleus pulposus cells (active nucleus pulposus cell phenotype).

In the present invention, particularly in the case where nucleus pulposus progenitor cells are obtained in the induction step and then nucleus pulposus cells (preferably, an active nucleus pulposus cell phenotype) are further obtained from the nucleus pulposus progenitor cells, a step of checking the expression status of a gene or a protein that characterizes the induced progenitor cells, that is, a nucleus pulposus cell marker in the cells during culture or after culture in the induction step (checking step 4) may be performed, as required.

Examples of the positive marker in induced nucleus pulposus cells include CD24, aggrecan, type II collagen, keratin 8, and keratin 18. According to one embodiment of the present invention, the cells during culture or after culture in the induction step, that is, (the cells estimated to be) induced nucleus pulposus cells preferably express (being positive in) at least one selected from the group consisting of CD24, aggrecan, and type II collagen, more preferably express (being positive in) all these three types.

Examples of the negative marker in induced nucleus pulposus cells include type I collagen. According to one embodiment of the present invention, the cells during culture or after culture in the differentiation induction step, that is, (the cells estimated to be) induced nucleus pulposus cells preferably do not express (being negative in) or weakly express type I collagen.

### Checking Step 2

Checking step 2 is a step of checking whether or not the cells during culture or after culture in the induction step are capable of forming (particularly spherical) colony-forming units under colony-forming assay culture conditions. The “colony-forming assay culture conditions” are known, and the same embodiment can he applied also to the present invention. Examples thereof include culture in a methylcellulose-containing medium. As one of the indices for determining that nucleus pulposus progenitor cells have been obtained by the induction step, the cells during culture or after culture in the induction step may be subjected in checking step 2, to confirm that colony-forming units are formed when the cells are cultured under colony-forming assay culture conditions.

### Checking Step 3

Checking step 3 is a step of checking whether or not the cells during culture or after culture in the induction step are capable of differentiating into a nucleus pulposus cell (preferably, active nucleus pulposus cell) phenotype or a notochord cell phenotype. The process for differentiation induction from nucleus pulposus progenitor cells into a nucleus pulposus cell phenotype or a notochord cell phenotype (such as a culture method and culture conditions) is known (for example, see Non Patent Literature 1 above). As one of the indices for determining that nucleus pulposus progenitor cells have been obtained by the induction step, the cells during culture or after culture in the induction step may be subjected to checking step 3, to confirm that nucleus pulposus cells are obtained when the process for differentiation induction from nucleus pulposus progenitor cells into a nucleus pulposus cell phenotype or a notochord cell phenotype is applied, in other words, the cells after the processes have an expression profile of a predetermined cell marker indicating that the cells are nucleus pulposus cells, by the method as described in this description.

In addition to checking step 3 or instead of checking step 3, a step of checking whether or not the cells during culture or after culture in the induction step are capable of differentiating into at least one selected from the group consisting of chondrocytes, adipocytes, and osteocytes may be performed. The process for differentiation induction from nucleus pulposus progenitor cells into nucleus pulposus cells or cells other than nucleus pulposus cells such as chondrocytes (such as a culture method and culture conditions) is known (for example, see Non Patent Literature 1 above).

Transdifferentiation or differentiation induction from cells into nucleus pulposus progenitor cells (preferably, an active nucleus pulposus progenitor cell phenotype) requires transduction and expression of nucleus pulposus progenitor cell master regulator transcription factors into the cells. The production of induced nucleus pulposus progenitor cells can be ended when it can be confirmed that the transcription factors-introduced cell that are being cultured (the desired proportion of transcription factors-introduced cell in the cell population in the cell culture) have become nucleus pulposus progenitor cells (preferably, an active nucleus pulposus progenitor cell phenotype) by determination based on at least one of checking steps 1 to 3, preferably overall determination based on checking steps 1 and 2 or 1 to 3.

The method for producing induced nucleus pulposus progenitor cells of the present invention as described above enables nucleus pulposus progenitor cells (preferably, active nucleus pulposus progenitor cell phenotype) to be supplied substantially infinitely (inexhaustibly). Use of induced nucleus pulposus progenitor cells to be obtained by the present invention is not limited, but the cells can be used typically for obtaining induced nucleus pulposus cells by the production method described below, for administration to an intervertebral disc in methods for treating and preventing the intervertebral disc disorders described below, particularly, for preparing cell preparations used in such applications.

### Transcription Factors-Introduced Cells/Induced Nucleus Pulposus Progenitor Cells

Both the transcription factors-introduced cells and induced nucleus pulposus progenitor cells of the present invention are cells produced by the method for producing induced nucleus pulposus progenitor cells of the present invention. In this description, cells containing an effective amount of a gene of nucleus pulposus progenitor cell master regulator transcription factors or a product thereof, that is, cells just having nucleus pulposus progenitor cell master regulator transcription factors introduced thereinto, and cells that overexpress nucleus pulposus progenitor cell master regulator transcription factors due to culture after the introduction but are not yet induced into an active nucleus pulposus progenitor cell phenotype are referred to as “transcription factors-introduced cells”, whereas cells that are obtained through culturing the transcription factors-introduced cells and induced into a phenotype as nucleus pulposus progenitor cells, preferably, an active nucleus pulposus progenitor cell phenotype are referred to as “induced nucleus pulposus progenitor cells”, so as to distinguish the two. The embodiment of a cell population containing transcription factors-introduced cells and/or induced nucleus pulposus progenitor cells, and the embodiment of a cell culture containing such a cell population can be adjusted corresponding to their applications. For example, in the case where a cell population containing induced nucleus pulposus progenitor cells is used as a raw material for producing a cell preparation for treating or preventing an intervertebral disc disorder, it is desirable to set the ratio of induced nucleus pulposus progenitor cells in the cell population as high as possible (conversely, to set the ratio of transcription factors-introduced cells that have not transformed to induced nucleus pulposus progenitor cells as low as possible).

Further, according to one embodiment of the present invention, transcription factors-introduced cells, cell cultures, induced nucleus pulposus progenitor cells, or cell populations obtained by the present invention can be used in a method for screening for a medicine or a method for treating or preventing an intervertebral disc disorder in a vertebrate animal including a step of testing the effectiveness and safety in a subject, that is, can be used as an in-vitro test model serving as a scientific, diagnostic, or prognostic tool for evaluating the reaction of nucleus pulposus progenitor cells or patient-specific nucleus pulposus progenitor cells to dosing, factors, or other (environmental) conditions. Further, a methodology established by the present invention can be used, for example, for fabricating nucleus pulposus progenitor cells from patients with genetic defects, in order to evaluate the influence of the genetic defects on nucleus pulposus progenitor cell phenotypes, homeostasis, development and pathology of an intervertebral disc, and reactions to drugs. The embodiment based on such applications, for example, enables the effectiveness of specific drugs on individual patients to be evaluated before administration, or the effectiveness of a treatment for preventing toxic side effects in administration of induced nucleus pulposus progenitor cells to patients or suppressing medical cost to be evaluated, in an individualized medical approach.

### Pharmaceutical Composition and Cell Preparation

The pharmaceutical composition of the present invention contains the nucleus pulposus progenitor cell inducer of the present invention, that is, an effective amount of a gene (nucleic acid) of nucleus pulposus progenitor cell master regulator transcription factors or a product thereof (protein). Further, the cell preparation of the present invention contains induced nucleus pulposus progenitor cells obtained by introducing an effective amount of nucleus pulposus progenitor cell master regulator transcription factors into cells outside the body (in vitro or ex vivo). The pharmaceutical composition and the cell preparation can be used for treating and preventing spine-related diseases of humans and non-human vertebrates. The pharmaceutical composition of the present invention can be used in the form of so-called gene treatment for transforming cells present in the nucleus pulposus into nucleus pulposus progenitor cells (preferably, active nucleus pulposus progenitor cell phenotype) inside the body (in vivo or in situ). Meanwhile, the cell preparation of the present invention can be used as an effective supply source, which potentially recovers biomechanical characteristics of the spine, for reconstituting and recovering the structure of an intervertebral disc by being transplanted into a nucleus pulposus with a degenerated intervertebral disc through allogeneic transplantation, xenotransplantation, or autologous cell transplantation.

Examples of “spine-related diseases” that can be treated and prevented by administration of the pharmaceutical composition and the cell preparation of the present invention (or further by the method described below) include diseases that manifest disorders, degeneration, hernia, and the like of the intervertebral disc (nucleus pulposus) as symptoms, such as intervertebral disc disease of the lumbar regions or the cervical spine, intervertebral hernia, cervical spondylotic myelopathy, radiculopathy, spondylolysis/spondylolisthesis, lumbar spinal stenosis, lumbar degenerative spondylolisthesis, and lumbar degenerative scoliosis.

The amounts of nucleus pulposus progenitor cell master regulator transcription factors (nucleic acids or proteins) contained in the pharmaceutical composition of the present invention and induced nucleus pulposus progenitor cells contained in the cell preparation can be appropriately adjusted and are not specifically limited, as long as the therapeutic or prophylactic effects desired are obtained. For example, the content of induced nucleus pulposus progenitor cells in the cell preparation of the present invention can be an amount such that 1 to 10×10⁷ induced nucleus pulposus progenitor cells are administered per intervertebral disc in the case of a human is targeted or can be an equivalent amount to above (converted amount) in the case where a vertebrate animal other than humans is targeted.

The dosage forms of the pharmaceutical composition and the cell preparation of the present invention need only to enable delivery to a nucleus pulposus of an intervertebral disc serving as a target and can be, for example, injections, preferably, injections for local administration to the intervertebral disc (nucleus pulposus). The pharmaceutical composition and the cell preparation, for example, when prepared as injections, can contain pharmaceutically acceptable substances such as injection solvents, normal saline, culture liquids, other suitable solvents/dispersion media, additives, and the like, as required. Further, the pharmaceutical composition and the cell preparation each can be produced also as a kit including a syringe and a pharmaceutical agent to be used in combination, as required.

### Method for Treating and Preventing Spine-Related Disease

A first embodiment (which may be referred to as “first treatment and prevention method” in this description) of the method for treating and preventing a spine-related disease of the present invention includes transplanting or administering (a cell population containing) the induced nucleus pulposus progenitor cells of the present invention or the cell preparation of the present invention containing (a cell population containing) the induced nucleus pulposus progenitor cells in vivo so as to act on the intervertebral disc nucleus pulposus tissue. The phrase “so as to act on the intervertebral disc nucleus pulposus tissue” means that the embodiment of transplantation or administration is not specifically limited, as long as the induced nucleus pulposus progenitor cells or the like that have been transplanted or administered can reach the intervertebral disc nucleus pulposus tissue that is an affected area, so that the effects of treatment or prevention can be exerted. The phrase includes transplanting the induced nucleus pulposus progenitor cells or the like into the intervertebral disc nucleus pulposus tissue or the vicinity thereof or injecting the induced nucleus pulposus progenitor cells or the like so as to reach the affected area through blood vessels, for example.

A second embodiment (which may be referred to as “second treatment and prevention method” in this description) of the method for treating and preventing a spine-related disease of the present invention includes administering the nucleus pulposus progenitor cell inducer of the present invention or the pharmaceutical composition of the present invention containing the nucleus pulposus progenitor cell inducer in vivo so as to act on nucleus pulposus cells in an intervertebral disc. The phrase “so as to act on nucleus pulposus cells in an intervertebral disc” means that the embodiment of administration is not specifically limited, as long as the administered nucleus pulposus progenitor cell inducer or the like can be incorporated into the nucleus pulposus cells in the intervertebral disc to reactivate the cells, so that the effects of treatment or prevention can be exerted. The phrase includes administering the nucleus pulposus progenitor cell inducer or the like into the intervertebral disc nucleus pulposus tissue or the vicinity thereof in situ or injecting the nucleus pulposus progenitor cell inducer or the like so as to reach the affected area through blood vessels, for example.

The intervertebral disc (nucleus pulposus tissue) that is subjected to the action of the cell preparation of the first treatment and prevention method of the present invention or the like and the pharmaceutical composition or the like of the second treatment and prevention method is an intervertebral disc with degeneration, aging, other symptoms. In such an intervertebral disc with degeneration or the like, healthy nucleus pulposus cells decrease, and senescent cells and fibrous nucleus pulposus cells increase. The induced nucleus pulposus progenitor cells transplanted or administered by the first treatment and prevention method and the induced nucleus pulposus progenitor cells generated (reactivated) by the second treatment and prevention method can survive in an intervertebral disc microenvironment, which is acidic, hyperosmotic and hypoglycemic, thereby increasing the amount of aggrecan, type II collagen, and the like to be produced in the extracellular matrix, so that a spine-related disease such as intervertebral disc disorder can be treated or prevented. In the second treatment and prevention method of the present invention, nucleus pulposus progenitor cell master regulator transcription factors are introduced into senescent cells and fibrous nucleus pulposus cells contained in the intervertebral disc, so that those cells can be transdifferentiated and induced into nucleus pulposus progenitor cells (preferably, active nucleus pulposus progenitor cell phenotype).

The cell preparation or the like of the first treatment and prevention method of the present invention and the pharmaceutical composition or the like of the second. treatment and prevention method may be administered in an effective amount for exerting desired treatment or prevention effects. Such an effective amount can be appropriately adjusted depending on the dose per administration, the number of doses, and the administration interval (the number of doses within a certain period), in consideration of the embodiments of the cell preparation and the pharmaceutical composition or the like, the administration subject, the administration route, and the like. Both the first and second treatment and prevention methods can be performed on humans and non-human vertebrates.

### Method for Producing Induced Nucleus Pulposus Cells

The method for producing induced nucleus pulposus cells of the present invention includes a step of culturing induced nucleus pulposus progenitor cells in vitro, to induce differentiation into nucleus pulposus cells (preferably, an active nucleus pulposus cell phenotype) or mature the cells (referred to as “nucleus pulposus cell-inducing step” in this description). The nucleus pulposus cell-inducing step can be performed following the aforementioned induction step for induction of nucleus pulposus progenitor cells from transcription factors-introduced cells (hereinafter referred to as “nucleus pulposus progenitor cell-inducing step”). Techniques for differentiation induction from nucleus pulposus progenitor cells (preferably, an active nucleus pulposus progenitor cell phenotype) into nucleus pulposus cells (preferably, an active nucleus pulposus cell phenotype) or maturing the cells (such as a culture method and culture conditions, for example, including the composition of the medium, the culture period, and the like) are known. The nucleus pulposus cell-inducing step in the present invention can be carried out according to such a known method, for example, by changing the composition of the medium (such as the aforementioned growth factors or the like to be added) in the nucleus pulposus progenitor cell-inducing step, as required.

The cell population to be obtained by the method for producing induced nucleus pulposus cells of the present invention contains not only induced nucleus pulposus cells but also a certain proportion of induced nucleus pulposus progenitor cells that have produced the induced nucleus pulposus cells. The cell population and cell preparation as described above, which are defined to “contain induced nucleus pulposus progenitor cells” in the present invention may be a cell population and a cell preparation “containing induced nucleus pulposus progenitor cells and induced nucleus pulposus cells” as one embodiment, and the description herein may be appropriately read as such.

### EXAMPLES

### (1) Human Nucleus Pulposus Tissue

In conducting this study, collection and use of human tissue samples were examined and approved by the institutional ethics review committee of Tokai University School of Medicine. Surgically excised tissue materials collected only from patients which have provided their informed consent were used.

### (2) Tissue Collection, Cell Separation, and Growth Culture

Intervertebral disc tissues were obtained from patients undergoing surgery associated with intervertebral hernia, degenerative disc disease, or scoliosis. The tissues were collected in saline and examined visually to separate gelatinous nucleus pulposus tissues from degenerated nucleus pulposus or annulus fibrosus tissues. The collected samples were cryopreserved at about −196° C. in a sufficient amount of CellBanker (R) cryopreservation solution (Nippon Zenyaku Kogyo Co., Ltd., Japan) or subjected to cell separation. The tissues were finely chopped into 1 cm³ fragments to obtain human nucleus pulposus cell populations. Thereafter, the tissues were digested with TrypLE express (Gibco, USA) at 37° C. for 1 hour, followed by digestion with 0.25 mg/mL collagenase-P (F. Hoffmann-La Roche, Ltd,, Switzerland) for 2 hours. The cell suspension obtained was filtered, washed, and seeded in 10% fetal bovine serum αMEM (Thermo Fisher Scientific, Inc., USA) (unless otherwise noted) at 3,000 to 5,000 cells/cm², to be grown under 5% O₂ until further use. Peripheral blood mononuclear cells were separated from the collected peripheral blood and then seeded in 20% fetal bovine serum αMEM (Thermo Fisher Scientific, Inc., USA) at 20,000 cells/cm², to be grown under 21% O₂ for 2 weeks before use.

### (3) Microarray Assay

In preparation for microarray analysis, the nucleus pulposus cells were sorted through fluorescence-activated cell sorting by FACS Vantage cells (BD Biosciences, USA). The nucleus pulposus cells were detached with 0.25% (w/v) trypsin and 0.001% (w(v) ethylenediaminetetraacetic acid (EDTA) and then stained with anti-human disialoganglioside GD2 (GD2) (BD Pharmingen; 14; G2a) mAb for 30 minutes and then with FITC-conjugated anti-mouse Ig goat (BD Biosciences) at 4° C. for 30 minutes. After washing, the cells were stained with allophycocyanin-conjugated anti-human Tie2. (R&D Systems, Inc., clone 83715) mAb and PE-conjugated or biotinylated anti-human CD24 (BD Biosciences; clone ML5) mAb for 1 hour. Cell samples were centrifuged at 4° C. and 1200 rpm for 5 minutes, then washed, and classified into Tie2+/GD2+/CD24−, Tie2−/GD2+/CD24−/+, and Tie2−/GD2−/CD24+ populations, Further, the populations were simultaneously applied to methylcellulose medium MethoCult H4230 (STEMCELL Technologies Inc., USA), so as to be able to form spherical colony-forming units, and the Tie2−/GD2−/CD24+ population was obtained therefrom according to study by Sakai et al. (Sakai, D., Nakamura, Y., Nakai, T., Mishima, T., Kato, S., Grad, S., Mochida, J. (2012). Exhaustion of nucleus pulposus progenitor cells with ageing and degeneration of the intervertebral disc. Nat Commun, 3, 1264. doi: 10.1038/ncomms2226). The fractionated cells were lysed in RNeasy Mini Kit RNA buffer (QIAGEN, Netherlands), to isolate total RNA according to the manufacturer's instructions. Using the Law Input Quick Amp Labeling Kit One Color (Agilent Technology, Inc., USA), RNA was converted into Cy3-labeled cRNA, followed by treatment with SurePrint G3 Human GE 8×60K v2 microarray (Agilent Technology, Inc., USA) and Gene Expression Hybridization Kit (Agilent Technology, Inc., USA) and then evaluation with the software Feature Extraction 7 (Agilent Technology Inc., USA) via an Agilent DNA microarray scanner (G2565CA, Agilent Technology, Inc., USA). The expression profiles in the four different nucleus pulposus cell populations were compared by subtraction with the expression profiles in neural progenitor cells (ID GSM1608144, GSM1608145), fibroblasts (ID GSM1191059, GSM1191060, GSM1191061), iPS cells (ID GSM1598135, GSM1598136, GSM1598137), and lung cells (ID GSM1700910, GSM1700913) determined by database microarray obtained from the NCBI database (https://www.ncbi.nlm.nih.gov/gds/).

FIG. 1[B] to [D] show the results. Microarray assays revealed that over 150 transcription factors with overall high expression in the nucleus pulposus cell populations, out of a total of over 2000 transcription factors. The transcription factors arranged in descending order of expression level are as follows: (1) PAX1, (2) PITX1, (3) BARX1, (4) FOXQ1, (5) HOXC9, (6) HOXC4, (7) HOXA9, (8) HOXA6, (9) HOXB8, (10) PAX9, (11) PRRX1, (12) FOSB, (13) HOXB9, (14) HOXA3, (15) HOXA10, (16) HOXC6, (17) GLIS1, (18) EPAS1, (19) SIX1, (20) MKX, (21) HOXC8, (22) GATA6, (23) NKX3-2, (24) SIX2, (25) RUNX1, (26) HOXA7, (27) HOXB6, (28) HOXC10, (29) FOXC1, (30) HOXA5, (31) HOXD3, (32) FOXF2, (33) FOS, (34) ZFP36, (35) FOXL1, (36) KLF9, (37) SNA12, (38) TBX15, (39) KLF2, (40) NFIA, (41) PRRX2, (42) KLF4, (43) HOXB5, (44) PRDM8, (45) T, (46) VDR, (47) HOXA2, (48) HOXA11, (49) HOXB3, (50) NR4A2, (51) FOXF1, (52) FOSL2, (53) NFIX, (54) TLE2, (55) NR4A3, (56) NPAS2, (57) HSF4, (58) SIX4, (59) HOXB7, (60) HOXB2, (61) EGR1, (62) KLF8, (63) ZMAT3, (64) ZFHX4, (65) MYC, (66) SOX9, (67) NFIL3, (68) STAT2, (69) FOXC2, (70) GATA3, (71) PRDM16, (72) SMAD9, (73) SNAl1, (74) JUNB, (75) HOXD8, (76) DMRTA1, (77) SOX6, (78) PITX2, (79) PARG, (80) FOXS1, (81) FLl1, (82) FOSL1, (83) NFATC4, (84) FOXA1, (85) MAFB, (86) DLX3, (87) SOX5, (88) NR3C1, (89) PKNOX2, (90) VAX1, (91) LBX2, (92) HOXB4, (93) OSR1, (94) THZ3, (95) DLX5, (96) MEIS2, (97) GSC, (98) KLF5, (99) MSCs, (100) TBX18, (101) KLF10, (102) FOXP2, (103) GLIS3, (104) DBX1, (105) HOXC12, (106) DLX2, (107) HOXC5, (108) PLAGL1, (109) HOXC13, (110) HOXD10, (111) PAX6, (112) HNF4A, (113) NKX6-1, (114) ZBTB7B, (115) HLF, (116) TBX4, (117) MLXIPL, (118) NFATC1, (119) ID3, (120) ATF3, (121) ID2, (122) HOXC11, (123) HIVEP2, (124) PGR, (125) PPARA, (126) ZIC1, (127) MYBL1, (128) LHX9, (129) OSR2, (130) STAT1, (131) FEY, (132) TBX2, (133) TWIST1, (134) STAT6, (135) MAF, (136) STAT5A, (137) PAX8, (138) EGR2, (139) TOX, (140) EBF1, (141) FOXD4, (142) OTX1, (143) NFE2L3, (144) HOXD9, (145) ZEB1, (146) NKX3-1, (147) TSHZ2, (148) ELF4, (149) FOXP1, (150) EBF3, (151) DLX4, (152) TBX1, (153) KLF6, (154) SIX5, (155) TSHZ1, (156) HOXA4, (157) FOX01, (158) RUNX2, (159) DMRT1, (160) IRX3, (161) ETS1, (162) MXD4, (163) ZHX1, (164) HNF1A, (165) MEIS1, (166) RUNX3, (167) HAND2, (168) EGR3, (169) VAX2, (170) MEOX2, (171) HIF1 A, (172) BNC1, (173) ZFPM2, (174) HIC1, (175) MSX1, (176) SHOX2, (177) JUN, (178) CREB3L 1, (179) TBX3, (180) HOXA 13, (181) BACH2, (182) lD1, (183) STAT4, (184) SOX11, (185) IRX5, (186) HES5, (187) LHX1, (188) BCL6B, (189) NR2F2, (190) NR2F1, (191) NANOG, (192) FOXA2, and (193) FOXM1. MYC (cMyc as used herein) is at position 65, and its expression level in Tie2+GD2+ nucleus pulposus cells is comparatively high.

### (4) Plasmid Amplification and Virus Particle Production

A pMX-IRES-GFP vector with Brachyury (T) inserted and a pMXs-GW vector with the gene construct of each transcription factor candidate inserted were provided by courtesy of the iPS cell Research institute, Kyoto University, Japan. Each plasmid was amplified by cloning the plasmid into MAX Efficiency Stbl2 chemically competent cells (Invitrogen, USA) through heat shock at 42° C. for 45 seconds and subsequent overnight incubation in LB-broth (Miller) (Sigma-Aldrich Co. LLC, USA). On the next day, plasmid DNA was isolated and purified using the NucleoBond Xtra Midi kit (Takara Bio Inc., Japan) according to the manufacturer's instructions.

In order to produce virus particles, platinum-GP retroviral packaging cells were seeded in a DMEM high glucose medium supplemented with 10% FBS, 1% pyruvate, and 50 U/mL (50 μg/mL) penicillin/streptomycin at a density of 55×10³ cells/cm2 on a 0.1% gelatin-coated plate (Sigma-Aldrich Co. LLC, USA). The next day, according to study by Kitazawa et al. (Kitazawa, K. et al. OVOL2 Maintains the Transcriptional Program of Human Corneal Epithelium by Suppressing Epithelial-to-Mesenchymal Transition. Cell reports 15, 1359-1368, doi: 10.1016/j.celrep.2016.04.020 (2016)), each 2 mL of medium was supplemented with a total of 69 μL including 5.4 μL of FuGENE (Promega, USA), 600 ng (0.6 μL) of pLP/VSVG (Invitrogen, USA) (expression plasmid for vesicular stomatitis virus G protein as an alternative envelope), 1200 ng (1.2 μL) of a pMXs-GW plasmid or pMX-IRES-GFP vector DNA encoding a singular transgene, and 60 μL of Opti-MEM (Thermo Fisher Scientific, Inc., USA). 24 hours later, the medium was refreshed, and further 24 hours later, the medium was recovered and filtered with a 0.45 μm pore diameter filter unit (Sigma-Aldrich Co. LLC, USA), to recover virus particles. The virus particle suspension was directly applied to each study.

### (5) Transdifferendation of Human Mesenchymal Stromal Cells By “Spinfection”

### Part 1: Cell Morphology

Human mesenchymal stromal cells were obtained from a commercial vendor (Lonza, Switzerland) or by aspiration and subsequent culture of bone marrow during in-house orthopedic surgery. Bone marrow-derived mononuclear cells in bone marrow puncture fluid were seeded in a-minimal essential medium Eagle medium (Thermo Fisher Scientific, Inc., USA) supplemented with 20% (v/v) fetal bovine serum, 100 U/mL penicillin and 100 μg/mL streptomycin (Thermo Fisher Scientific, Inc., USA) at 1000 to 2000 cells/cm². The cells were held in a 21% O₂ incubator chamber at 37° C. for 1 week, to attach mesenchymal stromal cells to the plastic surface. Then, the medium was refreshed, and non-attached cell population was discarded. Subsequently, the attached cells were further grown for 1 week and then passaged with 0.25% (w/v) trypsin and 0.001% (w/v) ethylenediaminetetraacetic acid (EDTA).

The mesenchymal stromal cells obtained were seeded at 5.5×10³ cells/cm² on an uncoated 6-well plate (FUJIFILM Wako Pure Chemical Corporation, Japan) submerged in Dulbecco's modified Eagle's medium (Thermo Fisher Scientific, Inc., USA) containing 10% (v/v) fetal bovine serum, 100 U/mL penicillin, and 100 μg/mL streptomycin (Thermo Fisher Scientific, Inc., USA), to be attached overnight. The next day, the medium was replaced with 2 mL of Dulbecco's modified Eagle's medium (Thermo Fisher Scientific, Inc., USA) without fetal bovine serum. 500 μL of virus particle-containing medium was added to each well up to a volume of 2 mL and stipulated to receive only one, two, or three transgenes, or no transgenes at all, for each designated transgene. A volume of 2 mL was composed of and filled with Dulbecco's modified Eagle's medium (Thermo Fisher Scientific, Inc., USA) without fetal bovine serum, to give a final volume of 4 mL for each well. The SHAM control group was composed of cells that received 500 μL of virus particle medium produced so as to contain an enhanced green fluorescent protein expression vector. Subsequently, the medium was supplemented with 40 μg of polybrene (Santa Cruz Biotechnology, Inc., USA). Then, the cells were subjected to a centrifugal force of 800 G at 30° C. for 30 minutes. The virus particle medium was discarded, and the cells obtained were washed with excess phosphate buffered saline and supplemented with 0.1% (v/v) insulin-transferrin-selenium-ethanolamine solution (Thermo Fisher Scientific, Inc., USA), 100 U/mL penicillin, and 100 μg/mL streptomycin (Thermo Fisher Scientific, Inc., USA), 1% (v/v) fetal bovine serum, and 50 μM L-Ascorbyl Magnesium Phosphate (FUJIFILM Wako Pure Chemical Industries, Ltd., Japan). Thereafter, the cells were transferred to an incubation chamber under 2% O₂ and 37° C., and the medium was refreshed 2 to 3 times a week. Changes in cell morphology and changes in green fluorescence expression were tracked over time using an Olympus IX70 fluorescence microscope (Olympus Corporation, Japan).

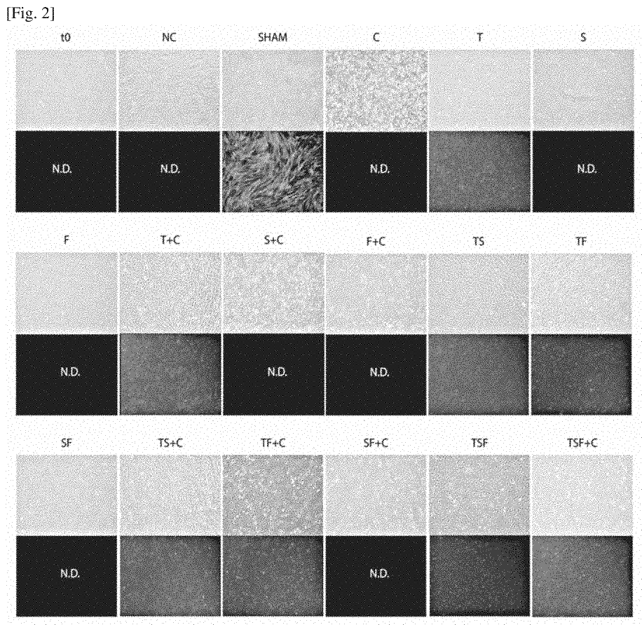

It was revealed that morphological changes in mesenchymal stromal cells cultured for 1 week after transduction were clear as compared with the same stromal cells on the day of transduction (t0), highly depending on the combination of transduced transgenes (FIG. 2). It was also revealed that, in the negative control (NC) condition and the SHAM control condition with transduction with the green fluorescent protein, morphological changes were limited, the morphology of fibroblasts showing thin elongated cells was maintained, and the proliferative capacity was high. Depending on the combination applied, addition of each transgene could change the cell morphology into a heterogeneous population, exhibiting polygonal cobblestone cells, polygonal cells characterized by dense cytoskeleton, elongated cells with long projections from the center, astrocytes-like cells having a plurality of projections like dendritic cells, or spherical cells appearing to be separated from the culture surface. Cells treated with cMyc exhibited more spherical cells with limited attachment to the plastic culture surface, as indicated by the presence of light surrounding the cells, referred to as “halos”. The combination of T and FOXQ1 (TF), T, FOXQ1, and cMyc (TF+C), T, FOXQ1, and SOX6 (TFS), or T, FOXQ1, SOX6, and cMyc (TFS+C) exhibited cells with a morphology similar to that of nucleus pulposus cells. The effectiveness of transduction was examined by the presence of intracellular green fluorescence, indicating that the green fluorescent protein was intracellularlly expressed, as an indicator of (i) successful transduction in SHAM control and (ii) successful transduction and expression of T in transduced cells containing T. The green fluorescence was detected in most of each transduction conditions containing T and/or in the SHAM control.

### (6) Transdifferentiation of Human Mesenchymal Stromal Cells By “Spinfection”

### Part 2: qPCR Analysis

Transduced cells under each conditions were recovered by incubation with 0.25% (w/v) trypsin and 0.001% (w/v) ethylenediaminetetraacetic acid (EDTA) and prepared for total ribonucleic acid (RNA) separation. Total RNA was separated using SV-Total RNA isolation System (Promega Corp., USA) according to the manufacturer's instructions. Subsequently, Capacity cDNA Reverse Transcription Kit (Applied Biosystem, USA) was applied, to convert the isolated RNA into complementary deoxyribonucleic acid (cDNA). Messenger ribonucleic acid (mRNA) expression profiles of the collected cells were evaluated by applying SYBR Green PCR Master Mix (Applied Biosystem, USA), a custom-designed primer (FASMAC Co. Ltd., Japan), and Quantstudio (R) 3 system (Applied Biosystems, USA). The resulting cycle threshold (CT) values were normalized to the internal control CT values obtained for glyceraldehyde 3-phosphate dehydrogenase expression and further normalized to the relative expression level of the non-transduced sample, and the relative expression level was calculated as 2-ΔΔCT.

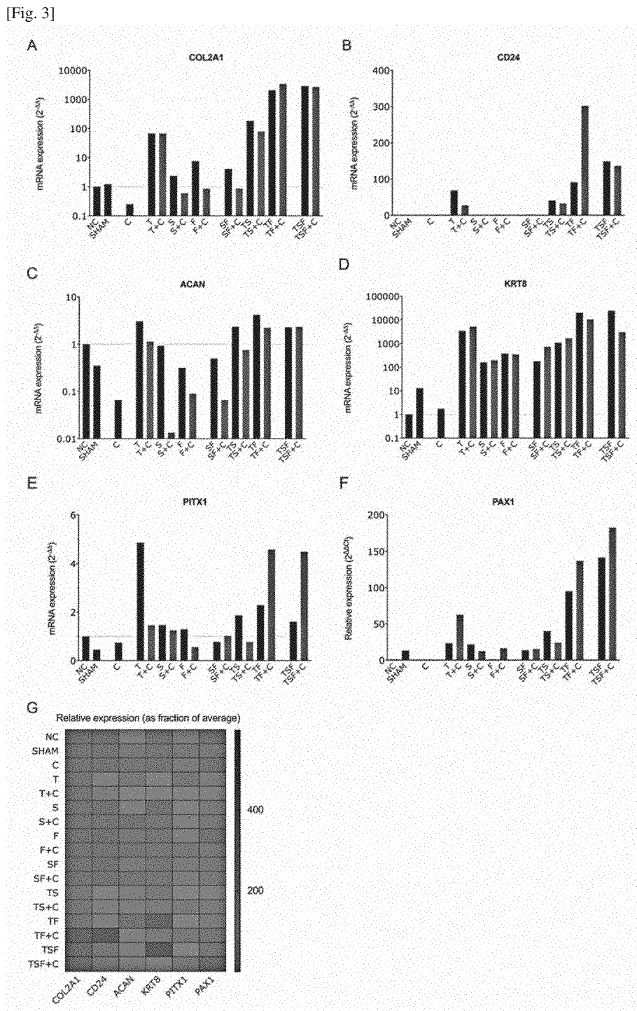

FIG. 3 shows the results obtained by qPCR analysis of mesenchymal stromal cells transduced with various combinations of T, SOX6, FOXQ1, and cMyc after the aforementioned culture for 2 weeks, as compared with mesenchymal stromal cells without transduction or transduced with green fluorescent protein (SHAM) (n=1). Relative mRNA expression levels of Collagen Type II alpha 1 (COL2A1), Paired Box 1 (PAX1), Cluster of Differentiation 24 (CD24), Paired-like homeodomain 1 (PITX1), Aggrecan (ACAN), and Keratin-8 (KRT8) were determined. The expression of COL2A1 significantly increased under all conditions with transduction with T and further amplified by the addition of FOXQ1. The expression of PAX1 (which has been shown to be the best transcription factor associated with the phenotype of nucleus pulposus cells and has been identified as a master regulator transcription factor for the phenotype of nucleus pulposus cells: see the description of PCT/JP2020/004449) showed the strongest increase among “T, FOXQ1, SOX6, and cMyc” (TES+C), “T, SOX6, and FOXQ1” (TSF), “T, FOXQ1, and cMyc” (TS+C), “T and FOXQ1” (TF), and PAX1 expression could be enhanced by the addition of cMyc for both combinations of “T and FOXQ1” (TF) and “T, SOX6 and FOXQ1” (TSF). Likewise, the expression of CD24 also strongly increased in “T, FOXQ1, SOX6, and cMyc” (TES+C), “T, SOX6, and FOXQ1” (TSF), “T, FOXQ1, and cMyc” (TF+C), indicating that the differentiation ability was enhanced by the addition of cMyc in mesenchymal stromal cells transduced with “T and FOXQ1” (TF). The increase in PITX1 could be enhanced only by the combination of “T” and maintained only by the addition of transcription factors FOXQ1 and cMyc. ACAN showed some increase in mesenchymal stromal cells transduced with the combinations of “T, FOXQ1, SOX6, and cMyc” (TFS+C), “T, SOX6 and FOXQ1” (TSF), “T, FOXQ1, and cMyc” (TF+C), “T and FOXQ1” (TF), and further “T and SOX6” (TS). Finally, the expression of KRT8 strongly increased in “T, SOX6 and FOXQ1” (TSF), “T, FOXQ1, and cMyc” (TF+C), and “T and FOXQ1” (TF). These results altogether suggest the ability of cMyc to enhance the expression of important nucleus pulposus cell markers.

### (7) Transdifferentiation of Human Mesenchymal Stromal Cells By “Spinfection”

### Part 3: Growth Rate and Colony Formation

Transduced mesenchymal stromal cells were obtained and cultured in 20% (v/v) fetal bovine serum nMEM (Thermo Fisher Scientific, Inc., USA) supplemented with 10 ng/mL basic fibroblast growth factor, 100 U/mL penicillin, and 100 streptomycin (Thermo Fisher Scientific, Inc., USA) under 5% O₂ tension. The transduced cells were cultured for about 3 weeks and carefully counted during passage.

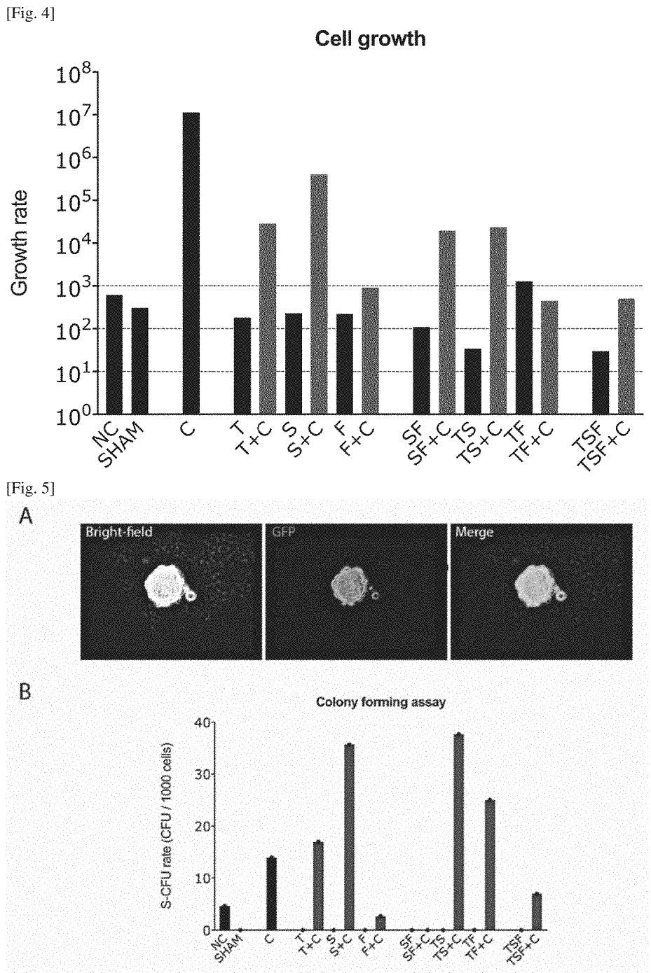

FIG. 4 suggests that the growth rate decreases under conditions of transduction with any one of T, SOX6, and FOXQ1, or combinations of these three, as compared with non-transduced cells or SHAM-transduced cells. The addition of cMyc to most combinations strongly increased the growth rate of the resulting cells.

Transduced cells obtained by culture in 20% (v/v) fetal bovine serum αMEM (Thermo Fisher Scientific, Inc., USA) supplemented with 10 ng/mL basic fibroblasts growth factor, 100 U/mL penicillin, and 100 μg/mL streptomycin (Thermo Fisher Scientific, Inc., USA) under 5% O₂ tension were subjected to a colony-forming assay by suspension culture in MethoCult H4230 methylcellulose medium (STEMCELL Technology, Canada), as described in study by Sakai et al. (Sakai et al., 2018). About 4,000 transduced cells were mixed with 4 mL of MethoCult H4230 methylcellulose medium (STEMCELL Technology, Canada) by vigorous manual shaking 1 mL of MethoCult cell suspension was transferred to a 35-mm diameter Petri dish and then cultured under 5% O₂ tension for 10 days. Temporarily, an inverted optical microscope was used, to track spheroid colonies and capture the images. An example of colonies formed on the 10th day is shown in FIG. 4. This example exhibits spherical colony-forming units consisting of more than 10 cells, which are mainly positive for the green fluorescent protein, suggesting active expression of T. The formation of colony-forming units suggests a stem cell-like or progenitor cell-like phenotype of the individual cells that produce the colony-forming units.

For all conditions analyzed, the number of spheroid colonies was counted with an inverted optical microscope. FIG. 5 shows average colony-forming units obtained by seeding 1000 cells in 1 mL of MethoCult H4230 methylcellulose medium (STEMCELL Technology, Canada), followed by culture for 10 days. The colony-forming unit rate could be evaluated only for samples with a sufficient number of living cells after growth for 1 week. As suggested by FIG. 4, the results for “SHAM”, “T” (T), “SOX6” (S), “FOXQ1” (F), “T and SOX6” (TS), “T and FOXQ1” (TF), “SOX6 and FOXQ1” (SF), “SOX6, FOXQ1, and cMyc” (SF+C), and “T, SOX6, FOXQ1, and cMyc” (TSF+C) are not shown in the colony-forming assay, since a sufficient number of cells could not be obtained in the first week of growth culture in conditions without addition of cMyc. Nevertheless, a high colony formation rate was observed in the conditions transduced with cMyc combined with one of the nucleus pulposus cell master regulator transcription factors, out of the conditions evaluated, as shown in FIG. 5. It is suggested again that cMyc can enhance the growth rate and. support or induce a stem cell-like or progenitor cell-like phenotype as well, so as to be capable of enabling colony formation in a heterogeneous population of transduced cells.

### (8) Transdifferentiation of Human Mesenchymal Stromal Cells By “Spinfection”

### Part 4: Changes in Cell Surface Markers

The transformed cells obtained by culture in 20% (v/v) fetal bovine serum αMEM (Thermo Fisher Scientific, Inc., USA) supplemented with 10 ng/mL basic fibroblast growth factor, 100 U/mL penicillin and 100 μg/mL streptomycin (Thermo Fisher Scientific, Inc., USA) under 5% O₂ tension were maintained for several weeks, and the expression of cell surface markers, cluster of differentiation 24 (CD24), disialoganglioside (GD2), and tyrosine--protein kinase (Tie2), and the general expression of the green fluorescent protein were analyzed over time by staining the samples with anti-CD24 antibody, anti-GD2 antibody, and anti-Tie2 antibody. The positive rate was analyzed by applying CellQuest Pro (BD Bioscience) and FlowJo Software (FlowJow LLC, Ashland, Oreg., US) via FACS Vantage (BD Bioscience, Erembodegem, Belgium).

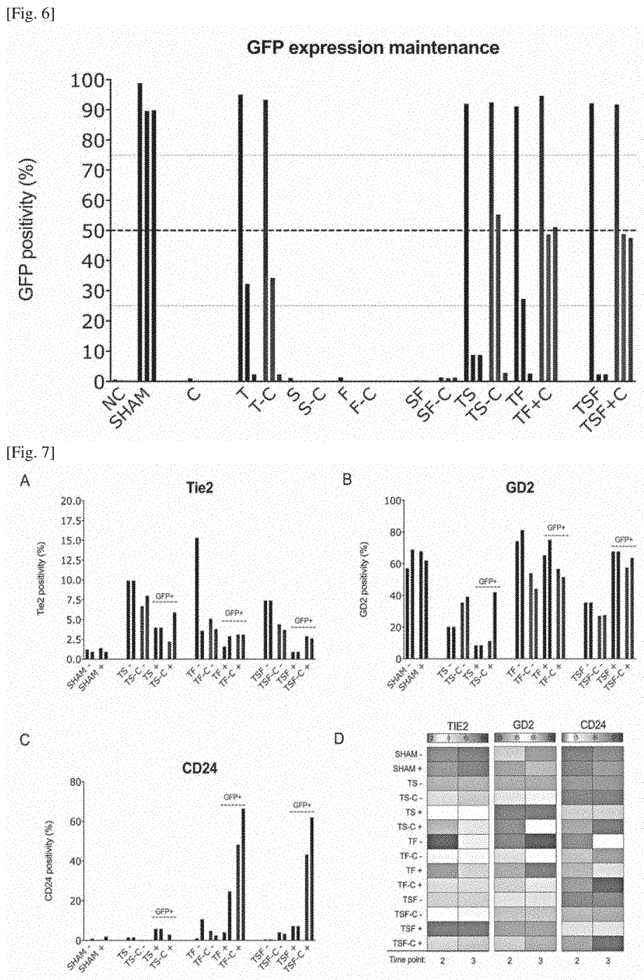

The positivity of the green fluorescent protein was evaluated as an indicator of T expression under all conditions with transduction with T (however, the positivity of the SHAM-transduced green fluorescent protein did not function as a T reporter). One time point indicates that there was a 1- to 2-week interval. FIG. 6 shows time-dependent changes in expression of the green fluorescent protein. At time point 1, all T-transduced conditions exhibited a positive rate of over 90%, indicating successful transduction of T vectors and active transcription. SHAM-transduced mesenchymal stromal cells could maintain expression of the green fluorescent protein at a level of over 90% during the observation period, suggesting that transcription of the vectors was not prevented, and/or mesenchymal stromal cells with no green fluorescent protein expression vectors obtained had a strong advantage. Under conditions without transduction with T or green fluorescent protein vectors, the positive rate of the green fluorescent protein was evaluated to be less than 1%, suggesting that the thresholds set to gate green fluorescent protein-positive cells were selective. Green fluorescent protein-positive cells reporting T expression could be maintained only under conditions containing the most potent nucleus pulposus cell master regulator transcription factors (see the description of PCT/JP2020/004449), that is, the combination of “T, SOX6, and FOXQ1” or “T and FOXQ1” with cMyc (TSF+C and TF+C). In other combinations without transduction with cMyc, that is, “T, SOX6, and FOXQ1” (TSF), “T and SOX6” (TS) and “T” and “T and FOXQ1” (TF) within a lesser range, the fractions of the green fluorescent protein-expressing cells drastically decreased at time point 2 and were completely lost at time point 3. The addition of cMyc to the combinations of “T and cMyc” (T+C) and “T, SOX6, and cMyc” (TS+C) could enhance the maintenance of the proportion of green fluorescent protein-expressing cells, but the enhancement was lost at time point 3. Overall, these results suggest that proliferative cells outnumbered the transduced cells that have lost expression of the green fluorescent protein (accordingly, T) due to interference of expression vectors or/and that did not express the green fluorescent protein (accordingly, T). Taken together, this data seems to suggest that master regulator transcription factors “T and FOXQ1” and SOX6 are preferable for enabling continuous expression of T, but the addition of cMyc is necessary for (1) enabling growth of nucleus pulposus cells induced by allowing a progenitor cell-like phenotype or/and (2) exerting the ability to prevent down-regulation of T expression vectors.

The transduced cells were cultured and obtained for analysis at a plurality of time points. The cells were subjected to flow cytometry analysis, to evaluate the Tie2-, GD2-, and CD24-positive rates of the transduced cells having various combinations of T, SOX6, FOXQ1, and cMyc in the green fluorescent protein-positive and -negative populations. FIG. 7[A] shows changes in Tie2-, GD2-, and CD24-positive rates for target primary transduction combinations. The expression of Tie2 was relatively high as compared with the SHAM control under each conditions, In the green fluorescent protein-positive population, the addition of cMyc tended to increase the Tie2-positive rate, regardless of the combinations of T, SOX6, and FOXQ1. Further, the addition of cMyc enabled the Tie2-positive rate to be maintained or enhanced from those at time point 2 and time point 3. In contrast, a higher Tie2-positive rate was observed overall in the green fluorescent protein-negative population, particularly as compared with SHAM. Interestingly, the addition of cMyc did not appear to enhance Tie2 expression, but rather exacerbated it, which suggests that the combination of T transduction and. expression (indicated by the positivity of the green fluorescent protein) and cMyc transduction may be particularly beneficial in enhancing Tie2 expression. Overall, the data seemed to suggest that cMyc can enhance the ability of the master regulator transcription factors described in the description of PCT/JP2020/004449 to induce the expression of Tie2, and the expression of the nucleus pulposus progenitor cell markers presented in Non Patent Literature 1 (Sakai et al., 2012) and subsequent Patent Literature 1 (International Publication No. WO 2011/122601).

As a result of evaluating the GD2 expression in the transduced cells, the differences between the conditions were limited (FIG. 7[B]). As compared with SHAM control, the absence of FOXQ1 and the introduction of SOX6 reduced the GD2 expression, which could be remedied by the introduction of FOXQ1 as long as T was expressed. For example, comparatively low GD2 expression was observed in the cells transduced with “T and SOX6” both with and without the green fluorescent protein expression (TS+ and TS−, respectively). The addition of cMyc could slightly enhance the expression level of GD2. However, the addition of FOXQ1 to this combination strongly increased GD2 expression, as indicated by the green fluorescence-positive combination of “T, SOX6, and FOXQ1” (TSF+), particularly under conditions where the green fluorescent protein was expressed. The advantages of FOXQ1 were further emphasized by the overall high expression of GD2 in the cells transduced with “T and FOXQ1.” (TF) and “T, FOXQ1, and cMyc” (TF+C).

As a result of evaluating CD24, the differences between different conditions were most remarkable (FIG. 7[C]). First, the expression of CD24 is mainly observed in green fluorescent protein expressing cells, suggesting a role for T in the induction of CD24. However, the combination containing the master regulator transcription factors particularly proposed in the description of PCT/JP2020/004449 resulted in a higher-positive rate of CD24, and the addition of cMyc could increase it more strongly. This suggests that eMyc has the ability to form a progenitor cell-like phenotype as highly dividing progenitor cells capable of producing mature CD24-expressing nucleus pulposus cells in a large amount, as presented in Non Patent Literature 1 (Sakai et al,, 2012) and subsequent Patent Literature 1 (International Publication No. WO 2011/122601).

### (9) Transdifferentiation of Human Mesenchymal Stromal Cells By “Spinfection”

### Part 5: Whole Tissue Culture

Transduced cells were obtained and sorted by the fluorescence-activated cell sorting technique, to obtain and prepare purified green fluorescent protein-expressing cells (excluding SHAM control, which serve as an indicator of T expression) for culture into nucleus pulposus tissue. At the same time, 0.38 grams of nucleus pulposus tissue fragments derived from a 19-year-old male patient previously obtained during spinal surgery and stored in liquid nitrogen storage was thawed on ice. Subsequently, nucleus pulposus tissue was irradiated with 150 kV of radiation at 20 mA and 1.64 Gy/min to be irradiated with 15 Gy of radiation, thereby removing living cells from the tissue. The cell-free tissue fragments were washed and macroscopically evenly divided for culturing the sorted green fluorescent protein-expressing cells obtained from SHAM-transduced cells, and “T, FOXQ1, and cMyc” (TF+C), “T, FOXQ1, SOX6, and cMyc” (TFS+C)-transduced mesenchymal stromal cells. The tissue fragments were transferred to a falcon tube, and 3 mL of 20% (v/v) fetal bovine serum αMEM (Thermo Fisher Scientific, Inc., USA) supplemented with 10 ng/mL basic fibroblast growth factor, 100 U/mL penicillin, and 100 μg/mL streptomycin (Thermo Fisher Scientific, Inc., USA) was added thereto. Then, each cells were seeded on the top of the tissue fragments and cultured under 5% O₂ tension. Using an Olympus IX70 fluorescence microscope. (Olympus Corporation, Japan), the presence and migration of green fluorescent protein-expressing cells were tracked in the tissue fragments.

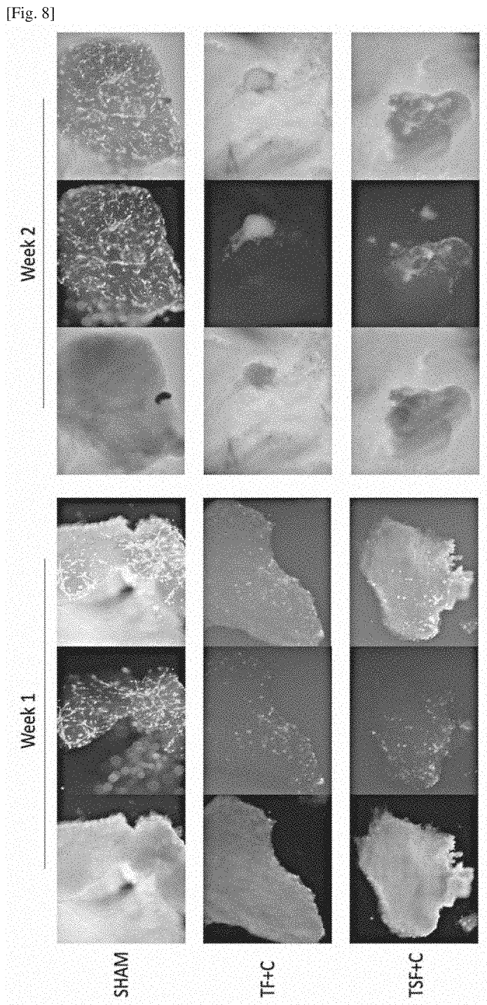

FIG. 8 shows the results. Imaging revealed that large green fluorescent protein-expressing cells were present, more fibroblast-like morphology was observed throughout the tissue fragments, and there was little change from the first week to the second week, under SHAM conditions. In both combinations of “T, FOXQ1, and cMyc” (TF+C) and “T, FOXQ1, SOX6, and cMyc” (TFS+C), sporadic rounded green fluorescent protein-expressing cells emerged mainly on the interaction surfaces of the tissue fragments, and then dense clusters occurred in the second week. This data suggests that “T, FOXQ1, and cMyc” (TF+C) and “T, FOXQ1, SOX6, and cMyc” (TES+C) migrated into the nucleus pulposus tissue fragments, then proliferated vigorously, and formed dense clusters of green fluorescent protein-expressing cells derived from the first sporadic green fluorescent protein-expressing cells. This data suggests proliferative, progenitor cell-like phenotypes of cells transduced with “T, FOXQ1, and cMyc” (TF+C) and “T, FOXQ1, SOX6, and cMyc” (TFS+C) and seeded on the nucleus pulposus tissue.

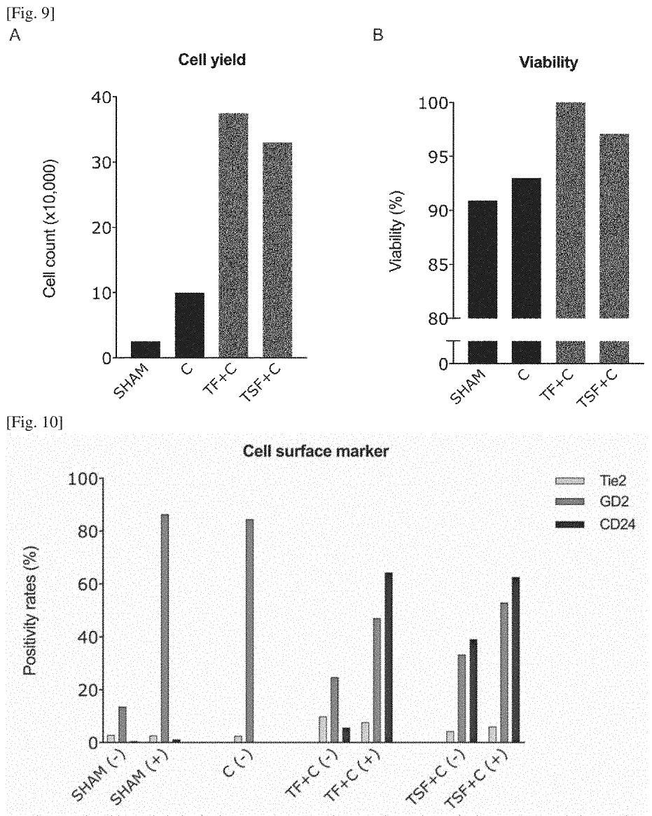

5 mL of TrypleExpress (Thermo Fisher Scientific, Inc., USA) was used at 37° C. for 30 minutes, followed by digestion by applying 0.0025 grams of collagenase (ROCHE) per 10 mL of 10% (v/v) fetal bovine serum αMEM (Thermo Fisher Scientific, Inc., USA) supplemented with 100 U/mL penicillin and 100 μg/mL streptomycin (Thermo Fisher Scientific, Inc., USA) at 37° C. for 60 minutes. Cells can be separated from the whole tissue culture in this way. Using the trypan blue exclusion test, the total cell yield for each conditions was examined by manual cell counting. FIG. 9[A] shows the total cell yield. This supports any one of a small number of cells of SHAM control, a slightly large number of cells of cMyc-only conditions, a significantly large number of cells transduced with “T, FOXQ1, and cMyc” (TF+C) and “T, FOXQ1, SOX6, and cMyc” (TFS+C), a higher proliferative, that is, progenitor cell-like phenotype, and a higher viability.

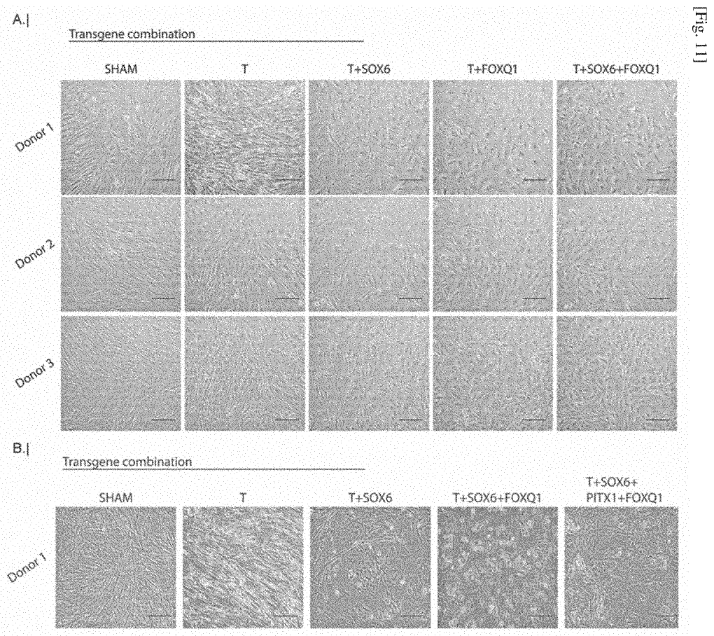

Subsequently, cells separated from the who tissue culture were subjected to flow cytometry analysis, to evaluate (1) cell viability (FIG. 9[B]) and (2) cell surface marker-positive rate (FIG. 10). As a result of examining the cell viability by propidium iodide staining, both SHAM conditions and cMyc conditions respectively exhibited comparatively low viabilities (about 90%) as compared with “T, FOXQ1, SOX6, and cMyc” (TSFA+C) and “T, FOXQ1, and cMyc” (TF+C), which viabilities are 97.1% and 100%, respectively. Combined with the total cell yield, it is clear that “T, FOXQ1, SOX6, and cMyc” (TFS+C) and “T, FOXQ1, and cMyc” (TF+C) exhibited far higher proliferation rates in the nucleus pulposus tissue than any one of SHAM and cMyc.

The evaluation of the cell surface markers revealed that cells transduced with “T, SOX6, FOXQ1, and cMyc” (TSF+C) and “T, FOXQ1, and cMyc” (TF+C) exhibited high expression of Tie2, regardless of whether they are positive for the green fluorescent protein. In contrast, SHAM- or cMyc-only conditions did not exhibit a high Tie2-positive fraction. The most remarkable is an increase in CD24-positive rate, which was mainly observed only in the green fluorescent protein-positive population ((+)) transduced with “T, SOX6, FOXQ1, and cMyc” (TSF+C) or “T, FOXQ1, and cMyc” (TF+C). The idea that “T, SOX6, FOXQ1, and cMyc” (TSFA+C) and “T, FOXQ1, and cMyc” (TFA+C) have the ability to produce CD24-expressing nucleus pulposus cells in a large amount, which is similar to the results obtained in Non Patent Literature 1 (Sakai et al., 2012) and subsequent Patent Literature 1 (International Publication No. WO 2011/122601), is promoted again. Further, these findings cumulatively support that nucleus pulposus progenitor cells or nucleus pulposus progenitor cell-like cells are preferred over mesenchymal stromal cell (MSCs) in intervertebral cell therapy products. This is because undifferentiated SHAM MSC samples have limited proliferative capacity in the nucleus pulposus tissue and are more incapable of differentiating into chondrocyte-like phenotype (as suggested by the lack of CD24 expression).

### Reference Examples

From Reference Examples below, those skilled in the art would be able to understand that the actions and effects of the present invention would be exerted, even in the case of using various terminally differentiated somatic cells such as fibroblasts.

### (10) Transdifferentiation of Human Fibroblasts: Part 1

Human newborn skin fibroblasts (Lonza, Switzerland) were grown in DMEM containing 10% (v/v) FBS, 100 U/mL penicillin, and 100 μg/mL streptomycin. The day before transduction, human fibroblasts were separated by incubation in 0.25% (w/v) trypsin, 0.001% (w/v) ethylenediaminetetraacetic acid (EDTA), and PBS for 5 minutes and then seeded at a density of 5.5×10³ cells/cm² in a well plate sufficiently covered with DMEM containing 6100 μg/mL penicillin and 100 μg/mL streptomycin.

To each well, 500 μL of viral culture medium transduced with each transgene was added in a maximum volume of 2 ml. As the SHAM control, a GFP transgene vector was added. DMEM containing 100 U/mL penicillin and 100 μg/mL streptomycin was added in a total capacity per well of 4 ml. Further, the culture medium was supplemented with 40 μg of polybrene (Santa Cruz Biotechnology, Inc.). The cultures were spun down at 30° C. and about 800 G for 30 minutes. Subsequently, the virus-containing culture medium was removed, and the cells were washed with excess PBS. Finally, the cells were cultured at 37° C. under 2% O₂ for two weeks in DMEM containing 0.1% (v/v) insulin-transferrin-selenium-ethanolamine solution (ITS-X; Thermo Fisher Scientific, Inc.), 1% (v/v) PBS, 50 μM L-magnesium ascorbyl phosphate n-hydrate (FUJIFILM Wako Pure Chemical Corporation, Japan), 100 U/mL penicillin and 100 μg/mL streptomycin, with or without supplementation with 10 ng/mL TGFβ1 (PeproTech, Inc., Japan) and 100 ng/mL growth differentiation factor 5 (GDF5 PeproTech, Inc.), and a fresh culture medium was given every 3 to 4 days. The cell morphology was captured by an optical microscope, and temporal changes were revealed depending on the combinations of transcription factors used for transduction.

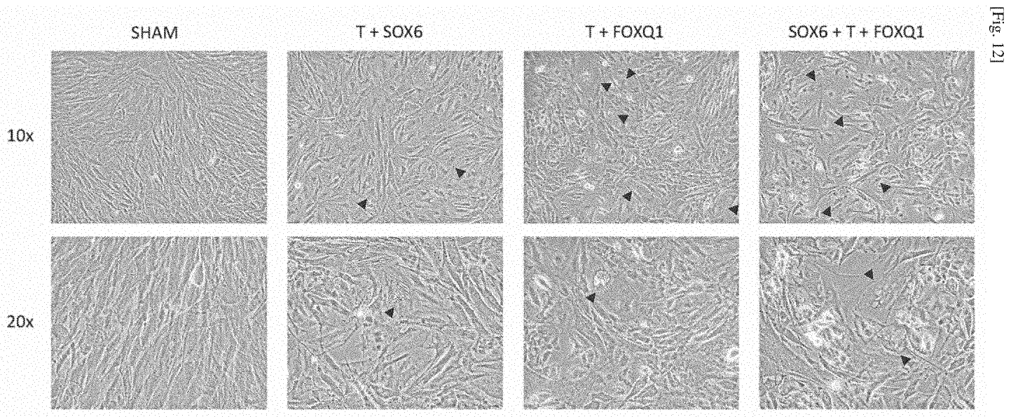

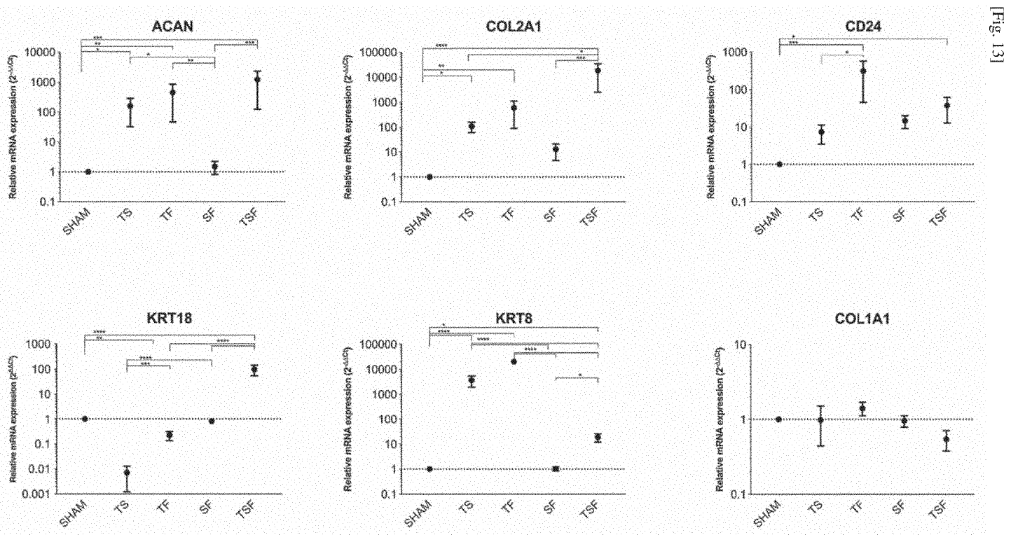

FIG. 11 and FIG. 12 show the results. Untreated, SHAM, or T-transduced fibroblasts showed little change while maintaining their elongated morphology throughout the differentiation culture (FIG. 11[A]). In contrast, transduction with a combination of two or three selected from T, SOX6, and FOXQ1 consistently resulted in a heterogeneous population with small fractions of cells maintaining the morphology of fibroblasts. Further, it seems from microscopic observation that the size of the cells increases, and the growth tendency is lost. Morphologically, the cells could be divided into three subpopulations: (i) cells having an elongated cell shape with the presence of long processes from the center, (ii) polygonal cells presenting strong cytoskeletal deposition, and (iii) astrocytic morphology presenting dendritic processes (FIG. 11[A]).

Finally, cells of a group transduced with transcription factors in combination sporadically presented cells with intercellular vacuoles similar to vacuoles observed in notochord cells in vitro (FIG. 12). Likewise, addition of PITX1 (data not shown) was accompanied by strong cytoskeletal deposition and increased cell size, strongly increasing the phenotype of (ii) above.

### (11) Transdifferentiation of Human Fibroblasts: Part 2

The transduced cells were collected with 0.25% trypsin and 0.001% EDTA and were subjected to further evaluation. For samples used for qPCR evaluation, total RNA was isolated using SV-Total RNA Isolation System (Promega Corp., USA) according to the manufacturer's instructions. The isolated RNA was thereafter converted into cDNA using a high-volume RNA-to-cDNA kit (Thermo Fisher Scientific, Inc.) according to the accompanying instructions. Subsequently, about 10 to 100 ng of cDNA was used for SYBRGREEN (Thermo Fisher Scientific, Inc.)-mediated qPCR analysis, and a custom-designed primer set for nucleus pulposus markers and notochord markers was applied. Each expression level obtained was calculated as a value of 2^{-ΔΔCT} comparing the gene expression level with that of GAPDH and subsequent SHAM control.

FIG. 13 shows the results. The qPCR analysis revealed a clear tendency of an increase in nucleus pulposus cell marker expression in the cells transduced with a combination of master regulator transcription factors identified, as compared with that in the SHAM-transduced cells. The expression profiles obtained showed an increase in mRNA expression levels of several selected nucleus pulposus cell markers for all combinations of double or triple transcription factors, T, FOXQ1, and SOX6 in fibroblasts one week after the transduction. These results revealed that transduction with a combination of T, SOX6, and FOXQ1 caused the most potent and the most consistent tendency to increase the extracellular matrix and the mRNA levels of aggrecan (ACAN) and type II collagen (COL2). Further, T+SOX6 and T+FOXQ1 showed a relatively higher KRT8 expression level as compared with the SHAM control, and transduction with triple transcription factors showed a strong tendency to increase the nucleus pulposus cell markers, KRT18 and KRT8. Further, most combinations showed a strong increasing tendency in CD24, and the combinations of T+SOX6+FOXQ1 and T+FOXQ1 were the highest. Further data (not included herein) showed that TSF and other combinations tend to improve the expression levels of PITX1, ANXA3, and OVOS. Finally, a negative marker, COL1A1, showed a desirable decreasing tendency only for T+SOX6+FOXQ1.

### (12) Transdifferentiation of Human Fibroblasts: Part 3

Further, two weeks after monolayer differentiation culture, transduced fibroblasts were counted as above at a density of 250,000 cells put into a 15 ml polypropylene conical tube (BD Biosciences) in 0.5 ml of the aforementioned differentiation culture medium. The cell suspension was spun down at 1500 rpm for 5 minutes at room temperature. After the cell pellets obtained were cultured for one day, the pellets were gently tapped from the bottom of the conical tube, and the spherical cell aggregates were further cultured under 2% O₂ for three weeks. Finally, the pellets were fixed with 4% (v/v) paraformaldehyde, supplemented with Tissue-TEK O.C.T. compound (Sakura Finetek Japan Co., Ltd., Japan), and rapidly frozen in liquid nitrogen. The sample obtained was cryosectioned into an 8 μm section on a silane-coated slide (MUTO pure chemical substance). Subsequently, production of ECM within a pellet was visualized by staining the section with a 1 g/L Safranin-O (Merck, USA) and 800 mg/L Fast Green FCF staining (Merck) solution or hematoxylin eosin staining.

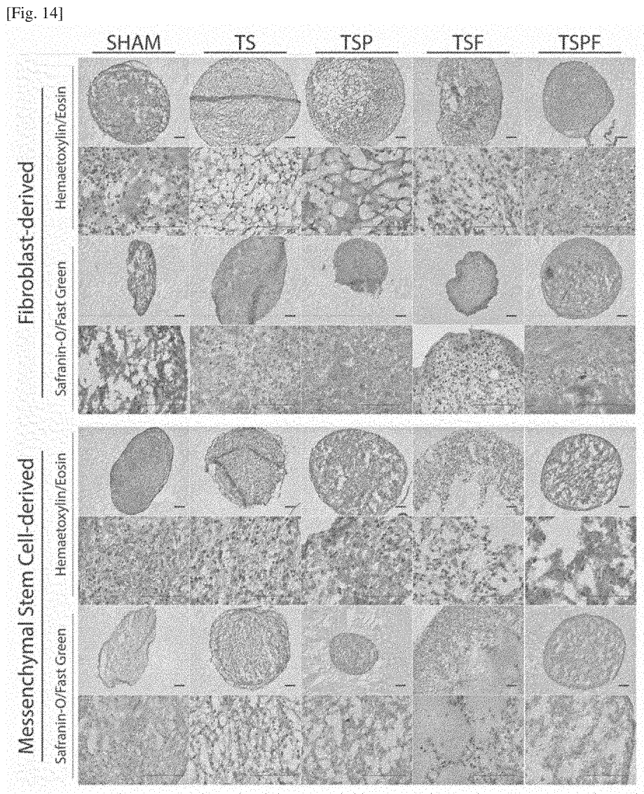

FIG. 14 shows the results. First, a pellet culture of fibroblasts transduced with GFP (SHAM) or a single transcription factor candidate (data not shown) generated a completely fibrous pellet structure without the presence of specific characteristics of a notochord or nucleus pulposus phenotype, which is shown by the lack of vacuolated cell morphology or the lack of proteoglycan deposition. in contrast, a pellet composed of fibroblasts transduced with a combination of transcription factors selected from SOX6, T, PITX1, and FOXQ1 showed a strong change in cell morphology toward a notochord phenotype and presented large vacuoles in the cytoplasm. Further, a pellet composed of fibroblasts transduced with T+S0X6, particularly T+SOX6+FOXQ1 showed mildly to intensely safranin-O stained regions and showed proteoglycan deposition throughout the notochord cell-like region within the pellet. The appearance of vesicles that are found in notochord cells indicates that differentiation from fibroblasts into a nucleus pulposus cell line was successful. This is because these characteristics cannot be generally seen in other mammalian cell types. Further, the presence of proteoglycans stained with safranin-O shows effective activation of the chondrogenic characteristics of a nucleus pulposus cell phenotype, indicating that there is a tendency of the cells to differentiate into a more mature nucleus pulposus cell phenotype beyond a notochord cell phenotype within the pellet culture.

1. A nucleus pulposus progenitor cell inducer that is an agent comprising an effective amount of genes of transcription factors for induction of a nucleated cell other than a nucleus pulposus progenitor cell into a nucleus pulposus progenitor cell (hereinafter referred to as “nucleus pulposus progenitor cell master regulator transcription factors”) or products thereof (hereinafter referred to as “nucleus pulposus progenitor cell inducer”), wherein
the nucleus pulposus progenitor cell master regulator transcription factor comprises Brachyury (T) or a homolog thereof, at least one selected from the group consisting of SRY-box6 (SOX6) or a homolog thereof and Forkhead Box Q1 (FOXQ1) or a homolog thereof, and MYC Proto-Oncogene, BHLH Transcription Factor (cMyc) or a homolog thereof.

2. The nucleus pulposus progenitor cell inducer according to claim 1, wherein the nucleus pulposus progenitor cell master regulator transcription factors are in the form of genes inserted into an expression vector(s).

3. A pharmaceutical composition for use in treating or preventing a spine-related disease in a vertebrate animal, comprising the nucleus pulposus progenitor cell inducer according to claim 1.

4. A method for producing an induced nucleus pulposus progenitor cell, comprising the steps of:
introducing the nucleus pulposus progenitor cell inducer according to claim 1 in vitro into a nucleated cell other than a nucleus pulposus progenitor cell (hereinafter referred to as “introduction step”); and
performing induction into the nucleus pulposus progenitor cell through culturing the cell obtained by the introduction step (hereinafter referred to as “transcription factors-introduced cell”) (hereinafter referred to as “induction step”).

5. The method for producing an induced nucleus pulposus progenitor cell according to claim 4, further comprising a step of checking an expression status of at least one selected from the group consisting of Tie2, GD2, and CD24 in the cell during culture or after culture in the induction step.

6. The method for producing an induced nucleus pulposus progenitor cell according to claim 4, further comprising a step of checking whether the cell during culture or after culture in the induction step is capable of forming a colony-forming unit under colony-forming assay culture conditions.

7. The method for producing an induced nucleus pulposus progenitor cell according to claim 4, further comprising a step of checking whether the cell during culture or after culture in the induction step is capable of differentiating into a nucleus pulposus cell.

8. The method for producing an induced nucleus pulposus progenitor cell according to claim 4, wherein the induction step comprises culturing the transcription factors-introduced cell in a medium supplemented with basic fibroblast growth factor (bFGF or FGF2), epidermal growth factor (EGF), or both of them.

9. The method for producing an induced nucleus pulposus progenitor cell according to claim 4, wherein the induction step comprises culturing the transcription factors-introduced cell under at least one condition selected from the group consisting of a hypoxic environment, an acidic environment, and a low glucose environment.

10. The method for producing an induced nucleus pulposus progenitor cell according to claim 4, wherein the induction step is performed under colony-forming assay culture conditions.

11. A transcription factors-introduced cell that is a cell comprising an effective amount of the nucleus pulposus progenitor cell master regulator transcription factors defined in claim 1.

12. An induced nucleus pulposus progenitor cell that is a cell having an active nucleus pulposus progenitor cell phenotype obtained through culturing the transcription factors-introduced cell according to claim 11.

13. The induced nucleus pulposus progenitor cell according to claim 12, wherein the induced nucleus pulposus progenitor cell is expressing at least one selected from the group consisting of Tie2, GD2, and CD24.

14. The induced nucleus pulposus progenitor cell according to claim 12, wherein the induced nucleus pulposus progenitor cells is capable of differentiating into at least one mature cell phenotype selected from a nucleus pulposus cell phenotype and a notochord cell phenotype.

15. A cell population comprising the transcription factors-introduced cell(s) according to claim 11.

16. A cell preparation for use in treating or preventing a spine-related disease in a vertebrate animal, comprising the induced nucleus pulposus progenitor cell(s) according to the cell population of claim 15.

17. A method for treating or preventing a spine-related disease in a vertebrate animal, comprising transplanting or administering the cell population according to claim 15 in vivo so as to act on the intervertebral disc nucleus pulposus tissue.

18. A method for treating or preventing a spine-related disease in a vertebrate animal, comprising administering the nucleus pulposus progenitor cell inducer according to claim 1 in vivo so as to act on nucleus pulposus cells in an intervertebral disc.

19. A method for screening a medicine or a method for treating or preventing a spine-related disease in a vertebrate animal, comprising a step of testing effectiveness and safety in a subject using the transcription factors-introduced cell(s) according to claim 11.

20. A method for obtaining an indicator associated with aging, degeneration, or disease state of an isolated nucleus pulposus cell population, comprising measuring the expression level of the nucleus pulposus progenitor cell master regulator transcription factors defined in claim 1 in the nucleus pulposus cell population.

21. A method for producing an induced nucleus pulposus cell, comprising a step of performing differentiation induction in vitro into an active nucleus pulposus cell or maturing the cell through culturing the induced nucleus pulposus progenitor cell according to claim 12.

22. A kit comprising the nucleus pulposus progenitor cell inducer according to claim 1.
